(19) United States
(12) Patent Application Publication
(10) Pub. No.: US 2022/0296966 A1
(43) Pub. Date: Sep. 22, 2022

(54) Cross-Platform and Connected Digital Fitness System

(71) Applicant: Elo Labs, Inc., New York, NY (US)

(72) Inventors: Sami Asikainen, Oakville (CA); Riikka Tarkkanen, Oakville (CA); Nathanael Montgomery, Port Perry (CA)

(21) Appl. No.: 17/833,807

(22) Filed: Jun. 6, 2022

Related U.S. Application Data
(63) Continuation-in-part of application No. 16/927,940, filed on Jul. 13, 2020.
(60) Provisional application No. 63/197,260, filed on Jun. 4, 2021.
(60) Provisional application No. 62/872,766, filed on Jul. 11, 2019.

(51) Int. Cl. A63B 24/00

(52) U.S. Cl. CPC A63B 24/0075; A63B 24/0062; A63B 2220/806; A63B 2024/0071; A63B 2024/0068; A63B 2024/0065

(57) ABSTRACT

A system and method for tracking physical activity of a user performing exercise movements and providing feedback and recommendations relating to performing the exercise movements is disclosed. The method includes receiving a stream of sensor data in association with a user performing an exercise movement over a period of time, processing the stream of sensor data, detecting, using a first classifier on the processed stream of sensor data, one or more poses of the user performing the exercise movement, determining, using a second classifier on the one or more detected poses, a classification of the exercise movement and one or more repetitions of the exercise movement, determining, using a third classifier on the one or more detected poses and the one or more repetitions of the exercise movement, feedback including a score for the one or more repetitions, the score indicating an adherence to predefined conditions for correctly performing the exercise movement, and presenting the feedback in real-time in association with the user performing the exercise movement.

### CROSS REFERENCE TO RELATED APPLICATIONS

The present application claims priority, under 35 U.S.C. § 119, of U.S. Provisional Patent Application No. 63/197,260, filed Jun. 4, 2021 and entitled “Cross-Platform and Connected Digital Fitness,” and is a continuation-in-part of U.S. patent application Ser. No. 16/927,940, filed Jul. 13, 2020 and entitled “Interactive Personal Training System,” which claims the benefit of U.S. Provisional Patent Application No. 62/872,766, filed Jul. 11, 2019 and entitled “Exercise System including Interactive Display and Method of Use,” all of which are hereby incorporated by reference in their entirety.

### BACKGROUND

The specification generally relates to tracking physical activity of a user performing exercise movements and providing feedback and recommendations relating to performing the exercise movements. In particular, the specification relates to a system and method for actively tracking physical performance of exercise movements by a user, analyzing the physical performance of the exercise movements using machine learning algorithms, and providing feedback and recommendations to the user.

Physical exercise is considered by many to be a beneficial activity. Existing digital fitness solutions in the form of mobile applications help users by guiding them through a workout routine and logging their efforts. Such mobile applications may also be paired with wearable devices logging heart rate, energy expenditure, and movement pattern. However, they are limited to tracking a narrow subset of physical exercises such as cycling, running, rowing, etc. Also, existing digital fitness solutions cannot match the engaging environment and effective direction provided by personal trainers at gyms. Personal trainers are not easily accessible, convenient or affordable to many potential users. It is important for a digital fitness solution to address the requirements relating to personalized training, tracking physical performance of exercise movements, and intelligently providing feedback and recommendation to users that benefit and advances their fitness goals.

This background description provided herein is for the purpose of generally presenting the context of the disclosure.

### SUMMARY

The techniques introduced herein overcome the deficiencies and limitations of the prior art at least in part by providing systems and methods for tracking physical activity of a user performing exercise movements and providing feedback and recommendations relating to performing the exercise movements.

According to one innovative aspect of the subject matter described in this disclosure, a method for generating a recommendation of a next action for a user is provided. The method includes: receiving a selection of a fitness content provider from a user; capturing sensor data including a video in association with the user performing a workout routine based on content from the fitness content provider; analyzing, using a machine learning model, the captured sensor data including the video in association with the user performing the workout routine; presenting a feedback to the user in association with the workout routine; and generating a recommendation of a next action for the user.

According to another innovative aspect of the subject matter described in this disclosure, a system for providing feedback in real-time in association with a user performing an exercise movement is provided. The system includes: one or more processors; a memory storing instructions, which when executed cause the one or more processors to: receive a selection of a fitness content provider from a user; capture sensor data including a video in association with the user performing a workout routine based on content from the fitness content provider; analyze, using a machine learning model, the captured sensor data including the video in association with the user performing the workout routine; present a feedback to the user in association with the workout routine; and generate a recommendation of a next action for the user.

These and other implementations may each optionally include one or more of the following operations. For instance, the operations may include: sending a request via an application programming interface (API) of the fitness content provider to retrieve content responsive to receiving the selection of the fitness content provider from the user, and presenting the retrieved content in a user interface that natively matches that of the fitness content provider, the fitness content provider being a third-party service provider; processing the captured sensor data including the video in association with the user performing the workout routine, creating a condensed video based on processing the captured sensor data including the video, identifying a segment in the condensed video corresponding to an exercise movement, determining metadata based on analyzing the captured sensor data including the video, and attaching the metadata to identified segment in the condensed video. Additionally, these and other implementations may each optionally include one or more of the following features. For instance, the features may include: analyzing the captured sensor data including the video in association with the user performing the workout routine comprising identifying one or more of a number of repetitions of an exercise movement, a detected weight of an exercise equipment used in the exercise movement, a score indicating adherence to proper form, and user performance statistics in association with the user performing the workout routine; presenting the feedback to the user in association with the workout routine comprising generating a three dimensional representation of an avatar based on the user, translating user performance of the workout routine to a view of a heat map highlighting a part of a body on the avatar that was trained, and presenting the three dimensional representation of the avatar including the view of the heat map; the view of the heat map highlighting the part of the body on the avatar indicating whether the part of the body was undertrained, overtrained, or optimally trained; generating the recommendation of the next action for the user comprising sending the condensed to a personal trainer for review, and receiving the recommendation of the next action for the user from the personal trainer; the metadata including one or more of repetition count, detected equipment weight, adherence score for proper form, and performance statistics; the fitness content provider being one from a group of an independent personal trainer, a pure play digital fitness content provider, and a fitness company; and the recommendation of the next action for the user being an adaptive workout to balance development in one or more fitness areas.

Other implementations of one or more of these aspects and other aspects include corresponding systems, apparatus, and computer programs, configured to perform the various action and/or store various data described in association with these aspects. Numerous additional features may be included in these and various other implementations, as discussed throughout this disclosure.

The features and advantages described herein are not all-inclusive and many additional features and advantages will be apparent in view of the figures and description. Moreover, it should be understood that the language used in the present disclosure has been principally selected for readability and instructional purposes, and not to limit the scope of the subject matter disclosed herein.

### BRIEF DESCRIPTION OF THE DRAWINGS

The techniques introduced herein are illustrated by way of example, and not by way of limitation in the figures of the accompanying drawings in which like reference numerals are used to refer to similar elements.

FIG. 1A is a high-level block diagram illustrating one embodiment of a system for tracking physical activity of a user performing exercise movements and providing feedback and recommendations relating to performing the exercise movements.

FIG. 1B is a diagram illustrating an example configuration for tracking physical activity of a user performing exercise movements and providing feedback and recommendations relating to performing the exercise movements.

FIG. 2 is a block diagram illustrating one embodiment of a computing device including a personal training application.

FIG. 3 is a block diagram illustrating an example embodiment of a feedback engine 208.

FIG. 4 shows an example graphical representation illustrating a 3D model of a user as a set of connected keypoints and associated analysis results.

FIG. 5 shows an example graphical representation of a user interface for creating a user profile of a user in association with the interactive personal training device.

FIG. 6 shows example graphical representations illustrating user interfaces for adding a class to a user's calendar on the interactive personal training device.

FIG. 7 shows example graphical representations illustrating user interfaces for booking a personal trainer on the interactive personal training device.

FIG. 8 shows example graphical representations illustrating user interfaces for starting a workout session on the interactive personal training device.

FIG. 9 shows example graphical representations illustrating user interfaces for guiding a user through a workout on the interactive personal training device.

FIG. 10 shows example graphical representations illustrating user interfaces for displaying real time feedback on the interactive personal training device.

FIG. 11 shows an example graphical representation illustrating a user interface for displaying statistics relating to the user performance of an exercise movement upon completion.

FIG. 12 shows an example graphical representation illustrating a user interface for displaying user achievements upon completion of a workout session.

FIG. 13 shows an example graphical representation illustrating a user interface for displaying a recommendation to a user on the interactive personal training device.

FIG. 14 shows an example graphical representation illustrating a user interface for displaying a leaderboard and user rankings on the interactive personal training device.

FIG. 15 shows an example graphical representation illustrating a user interface for allowing a trainer to plan, add, and review exercise workouts.

FIG. 16 shows an example graphical representation illustrating a user interface for a trainer to review an aggregate performance of a live class.

FIG. 17 is a flow diagram illustrating one embodiment of an example method for providing feedback in real-time in association with a user performing an exercise movement.

FIG. 18 is a flow diagram illustrating one embodiment of an example method for adding a new exercise movement for tracking and providing feedback.

FIG. 19 shows an example graphical representation illustrating a user interface for displaying real time feedback on the interactive personal training device.

FIG. 20 shows an example graphical representation illustrating a user interface for displaying statistics relating to the user completion of an exercise workout session.

FIG. 21 shows another example graphical representation illustrating a user interface for displaying statistics relating to the user completion of an exercise workout session.

FIG. 22 shows another example graphical representation illustrating a user interface for displaying statistics relating to the user completion of an exercise workout session.

FIG. 23 shows an example graphical representation illustrating a user interface for displaying adaptive training changes.

### DETAILED DESCRIPTION

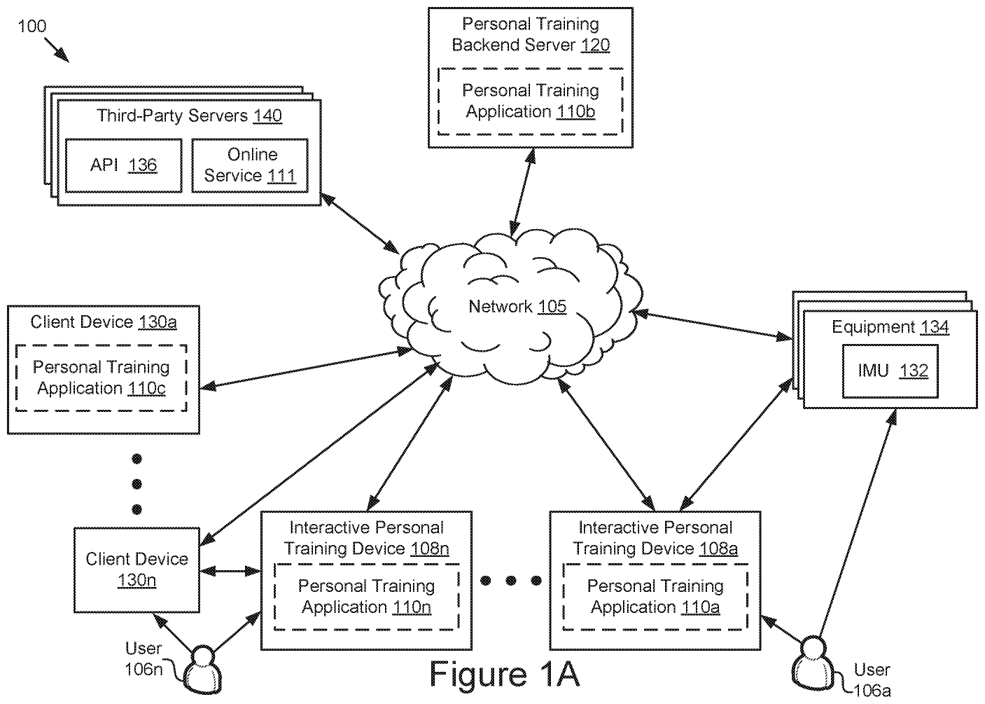

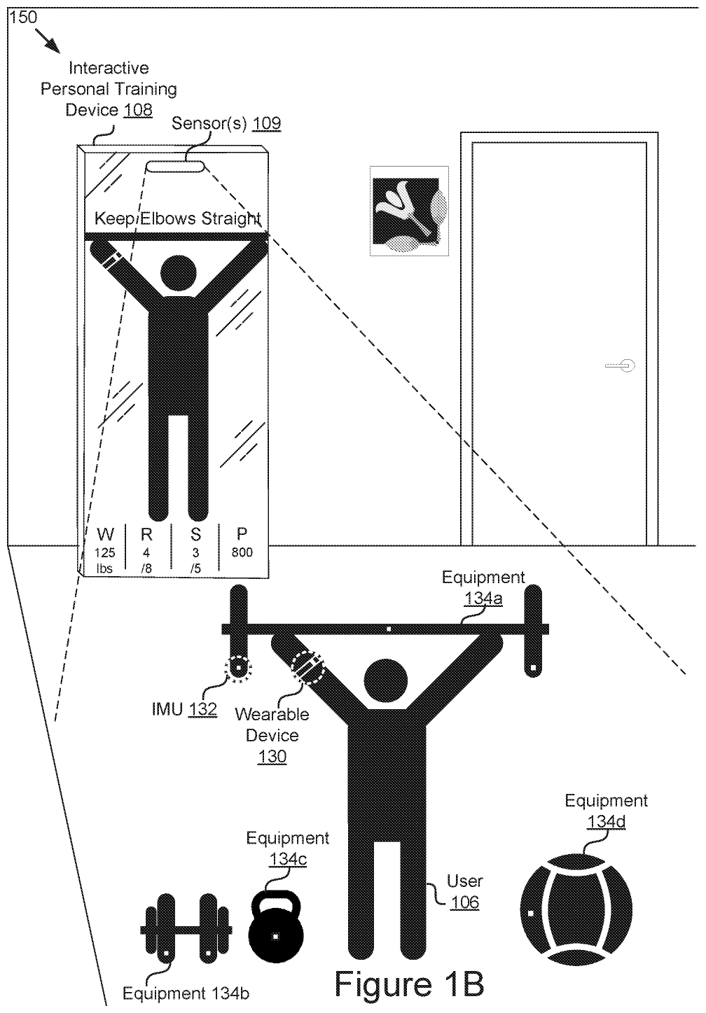

FIG. 1A is a high-level block diagram illustrating one embodiment of a system 100 for tracking physical activity of a user performing exercise movements and providing feedback and recommendations relating to performing the exercise movements. The illustrated system 100 may include interactive personal training devices 108a . . . 108n, client devices 130a . . . 130n, a personal training backend server 120, a set of equipment 134, and third-party servers 140, which are communicatively coupled via a network 105 for interaction with one another. The interactive personal training devices 108a . . . 108n may be communicatively coupled to the client device 130a . . . 130n and the set of equipment 134 for interaction with one another. In FIG. 1A and the remaining figures, a letter after a reference number, e.g., “108a,” represents a reference to the element having that particular reference number. A reference number in the text without a following letter, e.g., “108,” represents a general reference to instances of the element bearing that reference number.

The network 105 may be a conventional type, wired or wireless, and may have numerous different configurations including a star configuration, token ring configuration, or other configurations. Furthermore, the network 105 may include any number of networks and/or network types. For example, the network 105 may include a local area network (LAN), a wide area network (WAN) (e.g., the Internet), virtual private networks (VPNs), mobile (cellular) networks, wireless wide area network (WWANs), WiMAX® networks, Bluetooth® communication networks, peer-to-peer networks, and/or other interconnected data paths across which multiple devices may communicate, various combinations thereof, etc. The network 105 may also be coupled to or include portions of a telecommunications network for sending data in a variety of different communication protocols. In some embodiments, the network 105 may include Bluetooth communication networks or a cellular communications network for sending and receiving data including via short messaging service (SMS), multimedia messaging service (MMS), hypertext transfer protocol (HTTP), direct data connection, WAP, email, etc. In some implementations, the data transmitted by the network 105 may include packetized data (e.g., Internet Protocol (IP) data packets) that is routed to designated computing devices coupled to the network 105. Although FIG. 1A illustrates one network 105 coupled to the client devices 130, the interactive personal training devices 108, the set of equipment 134, the personal training backend server 120, and the third-party servers 140 in practice one or more networks 105 can be connected to these entities.

The client devices 130a . . . 130n (also referred to individually and collectively as 130) may be computing devices having data processing and communication capabilities. In some implementations, a client device 130 may include a memory, a processor (e.g., virtual, physical, etc.), a power source, a network interface, software and/or hardware components, such as a display, graphics processing unit (GPU), wireless transceivers, keyboard, camera (e.g., webcam), sensors, firmware, operating systems, web browsers, applications, drivers, and various physical connection interfaces (e.g., USB, HDMI, etc.). The client devices 130a . . . 130n may couple to and communicate with one another and the other entities of the system 100 via the network 105 using a wireless and/or wired connection. Examples of client devices 130 may include, but are not limited to, laptops, desktops, tablets, mobile phones (e.g., smartphones, feature phones, etc.), server appliances, servers, virtual machines, smart TVs, media streaming devices, user wearable computing devices (e.g., fitness trackers) or any other electronic device capable of accessing a network 105. In the example of FIG. 1A, the client device 130a is configured to implement a personal training application 110. While two or more client devices 130 are depicted in FIG. 1A, the system 100 may include any number of client devices 130. In addition, the client devices 130a . . . 130n may be the same or different types of computing devices. In some implementations, the client device 130 may be configured to implement a personal training application 110.

The interactive personal training devices 108a . . . 108n may be computing devices with data processing and communication capabilities. In the example of FIG. 1A, the interactive personal training device 108 is configured to implement a personal training application 110. The interactive personal training device 108 may comprise an interactive electronic display mounted behind and visible through a reflective, full-length mirrored surface. The full-length mirrored surface reflects a clear image of the user and performance of any physical movement in front of the interactive personal training device 108. The interactive electronic display may comprise a frameless touch screen configured to morph the reflected image on the full-length mirrored surface and overlay graphical content (e.g., augmented reality content) on and/or beside the reflected image. Graphical content may include, for example, a streaming video of a personal trainer performing an exercise movement. The interactive personal training devices 108a . . . 108n may be voice, motion, and/or gesture activated and revert back to a mirror when not in use. The interactive personal training devices 108a . . . 108n may be accessed by users 106a . . . 106n to access on-demand and live workout sessions, track user performance of the exercise movements, and receive feedback and recommendation accordingly. The interactive personal training device 108 may include a memory, a processor, a camera, a communication unit capable of accessing the network 105, a power source, and/or other software and/or hardware components, such as a display (for viewing information provided by the entities 120 and 140), graphics processing unit (for handling general graphics and multimedia processing), microphone array, audio exciters, audio amplifiers, speakers, sensor(s), sensor hub, firmware, operating systems, drivers, wireless transceivers, a subscriber identification module (SIM) or other integrated circuit to support cellular communication, and various physical connection interfaces (e.g., HDMI, USB, USB-C, USB Micro, etc.). In some implementations, the interactive personal training device 108 may be the client device 130.

The set of equipment 134 may include equipment used in the performance of exercise movements. Examples of such equipment may include, but not limited to, dumbbells, barbells, weight plates, medicine balls, kettlebells, sandbags, resistance bands, jump rope, abdominal exercise roller, pull up bar, ankle weights, wrist weights, weighted vest, plyometric box, fitness stepper, stair climber, rowing machine, smith machine, cable machine, stationary bike, stepping machine, etc. The set of equipment 134 may include etchings denoting the associated weight in kilograms or pounds. In some implementations, an inertial measurement unit (IMU) sensor 132 may be embedded into a surface of the equipment 134. In some implementations, the IMU sensor 132 may be attached to the surface of the equipment 134 using an adhesive. In some implementations, the IMU sensor 132 may be inconspicuously integrated into the equipment 134. The IMU sensor 132 may be a wireless IMU sensor that is configured to be rechargeable. The IMU sensor 132 comprises multiple inertial sensors (e.g., accelerometer, gyroscope, magnetometer, barometric pressure sensor, etc.) to record comprehensive inertial parameters (e.g., motion force, position, velocity, acceleration, orientation, pressure etc.) of the equipment 134 in motion during the performance of exercise movements. The IMU sensor 132 on the equipment 134 is communicatively coupled with the interactive personal training device 108 and is calibrated with the orientation, associated equipment type, and actual weight value (kg/lbs) of the equipment 134. This enables the interactive personal training device 108 to accurately detect and track acceleration, weight volume, equipment in use, equipment trajectory, and spatial location in three-dimensional space. The IMU sensor 132 is operable for data transmission via Bluetooth® or Bluetooth Low Energy (BLE). The IMU sensor 132 uses a passive connection instead of active pairing with devices, such as the client device 130, the interactive personal training device 108, etc. to improve data transfer reliability and latency. For example, the IMU sensor 132 records sensor data for transmission to the interactive personal training device 108 only when accelerometer readings indicate the user is moving the equipment 134. In some implementations, the equipment 134 may incorporate a haptic device to create haptic feedback including vibrations or a rumble in the equipment 134. For example, the equipment 134 may be configured to create vibrations to indicate to the user a completion of one repetition of an exercise movement as communicated to it by the personal training application 110 on one or more of the interactive personal training device 108, the client device 130, and the personal training backend server 120.

Also, instead of or in addition to the IMU sensor 132, the set of equipment 134 may be embedded with one or more of radio-frequency identification (RFID) tags for transmitting digital identification data (e.g., equipment type, weight, etc.) when triggered by an electromagnetic interrogation pulse from a RFID reader on devices, such as the client device 130 and the interactive personal training device 108 and machine-readable markings or labels, such as a barcode, a quick response (QR) code, etc. for transmitting identifying information about the equipment 134 when scanned and decoded by built-in cameras in the interactive personal training device 108 and the client device 130. In some other implementations, the set of equipment 134 may be coated with a color marker that appears as a different color in nonvisible light enabling the devices, such as the interactive personal training device 108 and the client device 130 to distinguish between different equipment type and/or weights. For example, a 20 pound dumbbell appearing black in visible light may appear pink to an infrared (IR) camera associated with the interactive personal training device 108.

Each of the plurality of third-party servers 140 may be, or may be implemented by, a computing device including a processor, a memory, applications, a database, and network communication capabilities. A third-party server 140 may be a Hypertext Transfer Protocol (HTTP) server, a Representational State Transfer (REST) service, or other server type, having structure and/or functionality for processing and satisfying content requests and/or receiving content from one or more of the client devices 130, the interactive personal training devices 108, and the personal training backend server 120 that are coupled to the network 105. In some implementations, the third-party server 140 may include an online service 111 dedicated to providing access to various services and information resources hosted by the third-party server 140 via web, mobile, and/or cloud applications. The online service 111 may obtain and store user data, content items (e.g., videos, text, images, etc.), and interaction data reflecting the interaction of users with the content items. User data, as described herein, may include one or more of user profile information (e.g., user id, user preferences, user history, social network connections, etc.), logged information (e.g., heart rate, activity metrics, sleep quality data, calories and nutrient intake data, user device specific information, historical actions, etc.), and other user specific information. In some embodiments, the online service 111 allows users to share content with other users (e.g., friends, contacts, public, similar users, etc.), purchase and/or view items (e.g., e-books, videos, music, games, gym merchandise, subscription, fitness products, fitness apparel, etc.), and other similar actions. For example, the online service 111 may provide various services such as physical fitness service; digital fitness service; digital fitness content provider; personal training; running and cycling tracking service; music streaming service; video streaming service; web mapping service; multimedia messaging service; electronic mail service; news service; news aggregator service; social networking service; photo and video-sharing social networking service; sleep-tracking service; diet-tracking and calorie counting service; ridesharing service; online banking service; online information database service; travel service; online e-commerce marketplace; ratings and review service; restaurant-reservation service; food delivery service; search service; health and fitness service; home automation and security service; Internet of Things (IOT), multimedia hosting, distribution, and sharing service; cloud-based data storage and sharing service; a combination of one or more of the foregoing services; or any other service where users retrieve, collaborate, and/or share information, etc. It should be noted that the list of items provided as examples for the online service 111 above are not exhaustive and that others are contemplated in the techniques described herein.

In some implementations, a third-party server 140 sends and receives data to and from other entities of the system 100 via the network 105. In the example of FIG. 1A, the components of the third-party server 140 are configured to implement an application programming interface (API) 136. For example, the API 136 may be a software interface exposed over the HTTP protocol by the third-party server 140. The API 136 includes a set of requirements that govern and facilitate the movement of information between the components of FIG. 1A. For example, the API 136 exposes internal data and functionality of the online service 111 hosted by the third-party server 140 to API requests originating from the personal training application 110 implemented on the interactive personal training device 108, the client device 130, and the personal training backend server 120. Via the API 136, the personal training application 110 passes an authenticated request including a set of parameters for information to the online service 111 and receives an object (e.g., XML, or JSON) with associated results from the online service 111. The third-party server 140 may also include a database coupled to the server 140 over the network 105 to store structured data in a relational database and a file system (e.g., HDFS, NFS, etc) for unstructured or semi-structured data. It should be understood that the third-party server 140 and the application programming interface 136 may be representative of one online service provider and that there may be multiple online service providers coupled to network 105, each having its own server or a server cluster, applications, application programming interface, and database.

In the example of FIG. 1A, the personal training backend server 120 is configured to implement a personal training application 110b. In some implementations, the personal training backend server 120 may be a hardware server, a software server, or a combination of software and hardware. In some implementations, the personal training backend server 120 may be, or may be implemented by, a computing device including a processor, a memory, applications, a database, and network communication capabilities. For example, the personal training backend server 120 may include one or more hardware servers, virtual servers, server arrays, storage devices and/or systems, etc., and/or may be centralized or distributed/cloud-based. Also, instead of or in addition, the personal training backend server 120 may implement its own API for the transmission of instructions, data, results, and other information between the server 120 and an application installed or otherwise implemented on the interactive personal training device 108. In some implementations, the personal training backend server 120 may include one or more virtual servers, which operate in a host server environment and access the physical hardware of the host server including, for example, a processor, a memory, applications, a database, storage, network interfaces, etc., via an abstraction layer (e.g., a virtual machine manager).

In some implementations, the personal training backend server 120 may be operable to enable the users 106a . . . 106n of the interactive personal training devices 108a . . . 108n to create and manage individual user accounts; receive, store, and/or manage functional fitness programs created by the users or obtained from third-party servers 140; enhance the functional fitness programs with trained machine learning algorithms; share the functional fitness programs with subscribed users in the form of live and/or on-demand classes via the interactive personal training devices 108a . . . 108n; and track, analyze, and provide feedback using trained machine learning algorithms on the exercise movements performed by the users as appropriate, etc. The personal training backend server 120 may send data to and receive data from the other entities of the system 100 including the client devices 130, the interactive personal training devices 108, and third-party servers 140 via the network 105. It should be understood that the personal training backend server 120 is not limited to providing the above-noted acts and/or functionality and may include other network-accessible services. In addition, while a single personal training backend server 120 is depicted in FIG. 1A, it should be understood that there may be any number of personal training backend servers 120 or a server cluster.

The personal training application 110 may include software and/or logic to provide the functionality for tracking physical activity of a user performing exercise movements and providing feedback and recommendations relating to performing the exercise movements. In some implementations, the personal training application 110 may be implemented using programmable or specialized hardware, such as a field-programmable gate array (FPGA) or an application-specific integrated circuit (ASIC). In some implementations, the personal training application 110 may be implemented using a combination of hardware and software. In other implementations, the personal training application 110 may be stored and executed on a combination of the interactive personal training devices 108, the client device 130, and the personal training backend server 120, or by any one of the interactive personal training devices 108, the client device 130 or the personal training backend server 120.

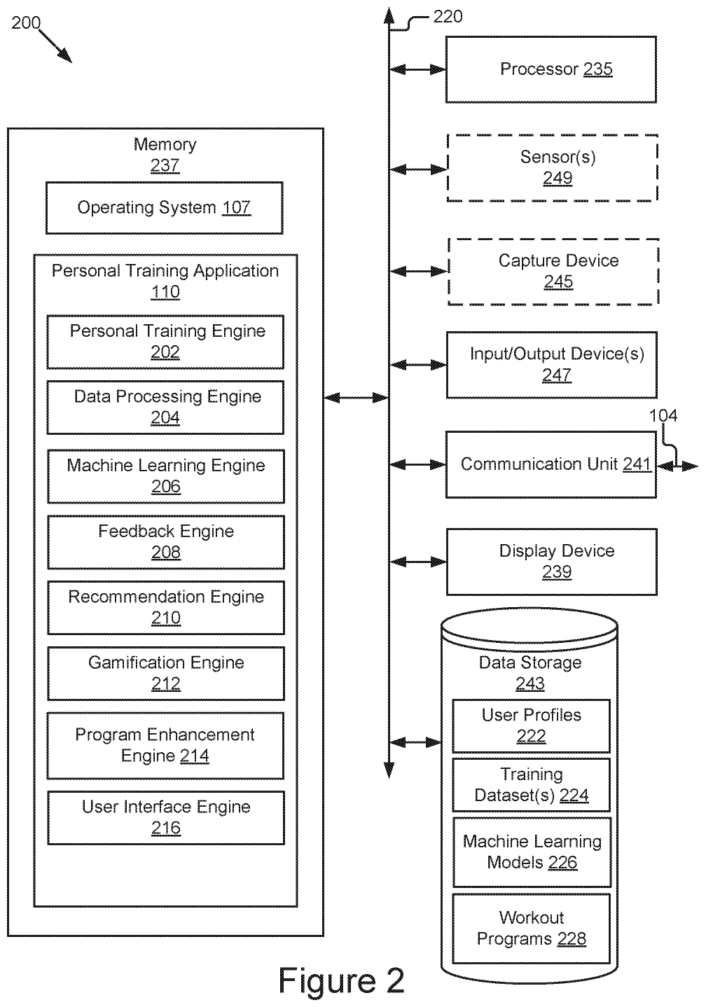

In some implementations, the personal training application 110 may be a thin-client application with some functionality executed on the interactive personal training device 108a (by the personal training application 110a) or on the client device 130 (by the personal training application 110c) and additional functionality executed on the personal training backend server 120 (by the personal training application 110b). For example, the personal training application 110a may be storable in a memory (e.g., see FIG. 2) and executable by a processor (e.g., see FIG. 2) of the interactive personal training device 108a to provide for user interaction, receive a stream of sensor data input in association with a user performing an exercise movement, present information (e.g., an overlay of an exercise movement performed by a personal trainer) to the user via a display (e.g., see FIG. 2), and send data to and receive data from the other entities of the system 100 via the network 105. The personal training application 110a may be operable to allow users to record their exercise movements in a workout session, share their performance statistics with other users in a leaderboard, compete on the functional fitness challenges with other users, etc. In another example, the personal training application 110b on the personal training backend server 120 may include software and/or logic for receiving the stream of sensor data input, analyzing the stream of sensor data input using trained machine learning algorithms, and providing feedback and recommendation in association with the user performing the exercise movement on the interactive personal training device 108. In some implementations, the personal training application 110a on the interactive personal training device 108a or the personal training application 110c on the client device 130 may exclusively handle the functionality described herein (e.g., fully local edge processing). In other implementations, the personal training application 110b on the personal training backend server 120 may exclusively handle the functionality described herein (e.g., fully remote server processing).

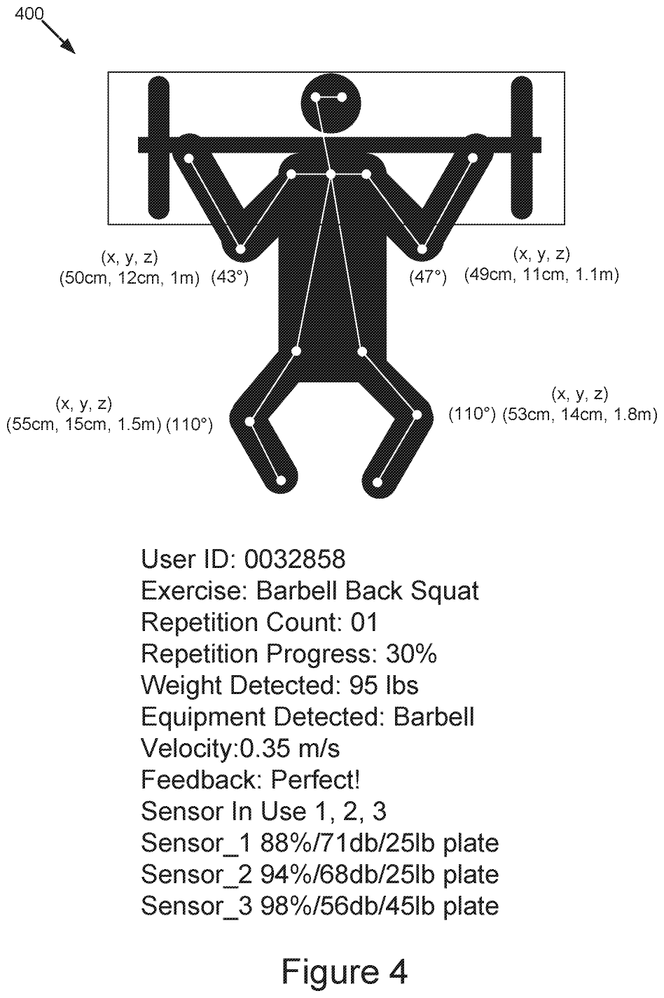

In some embodiments, the personal training application 110 may generate and present various user interfaces to perform these acts and/or functionality, which may in some cases be based at least in part on information received from the personal training backend server 120, the client device 130, the interactive personal training device 108, the set of equipment 134, and/or one or more of the third-party servers 140 via the network 105. Non-limiting example user interfaces that may be generated for display by the personal training application 110 are depicted in FIGS. 4-16 and 19-23. In some implementations, the personal training application 110 is code operable in a web browser, a web application accessible via a web browser on the interactive personal training device 108, a native application (e.g., mobile application, installed application, etc.) on the interactive personal training device 108, a combination thereof, etc. Additional structure, acts, and/or functionality of the personal training application 110 is further discussed below with reference to at least FIG. 2.

In some implementations, the personal training application 110 may require users to be registered with the personal training backend server 120 to access the acts and/or functionality described herein. For example, to access various acts and/or functionality provided by the personal training application 110, the personal training application 110 may require a user to authenticate his/her identity. For example, the personal training application 110 may require a user seeking access to authenticate their identity by inputting credentials in an associated user interface. In another example, the personal training application 110 may interact with a federated identity server (not shown) to register and/or authenticate the user by scanning and verifying biometrics including facial attributes, fingerprint, and voice.

It should be understood that the system 100 illustrated in FIG. 1A is representative of an example system for tracking physical activity of a user performing exercise movements and providing feedback and recommendations relating to performing the exercise movements, and that a variety of different system environments and configurations are contemplated and are within the scope of the present disclosure. For instance, various functionality may be moved from the personal training backend server 120 to an interactive personal training device 108, or vice versa and some implementations may include additional or fewer computing devices, services, and/or networks, and may implement various functionality client or server-side. Further, various entities of the system 100 may be integrated into to a single computing device or system or additional computing devices or systems, etc.

FIG. 1B is a diagram illustrating an example configuration for tracking physical activity of a user performing exercise movements and providing feedback and recommendations relating to performing the exercise movements. As depicted, the example configuration includes the interactive personal training device 108 equipped with the sensor(s) 109 configured to capture a video of a scene in which user 106 is performing the exercise movement using the barbell equipment 134a. For example, the sensor(s) 109 may comprise one or more of a high definition (HD) camera, a regular 2D camera, a RGB camera, a multi-spectral camera, a structured light 3D camera, a time-of-flight 3D camera, a stereo camera, a radar sensor, a LiDAR scanner, an infrared sensor, or a combination of one or more of the foregoing sensors. The sensor(s) 109 comprising of one or more cameras may provide a wider field of view (e.g., field of view >120 degrees) for capturing the video of the scene in which user 106 is performing the exercise movement and acquiring depth information (R, G, B, X, Y, Z) from the scene. The depth information may be used to identify and track the exercise movement even when there is an occlusion of keypoints while the user is performing a bodyweight exercise movement or weight equipment-based exercise movement. A keypoint refers to a human joint, such as an elbow, a knee, a wrist, a shoulder, hip, etc. The depth information may be used to determine a reference plane of the floor on which the exercise movement is performed to identify the occluded exercise movement. The depth information may be used to determine relative positional data for calculating metrics such as force and time-under-tension of the exercise movement. Concurrently, the IMU sensor 132 on the equipment 134a in motion and the wearable device 130 on the person of the user are communicatively coupled with the interactive personal training device 108 to transmit recorded IMU sensor data and recorded vital signs and health status information (e.g., heart rate, blood pressure, etc.) during the performance of the exercise movement to the interactive personal training device 108. For example, the IMU sensor 132 records the velocity and acceleration, 3D positioning, and orientation of the equipment 134a during exercise movement. Each equipment 134 (e.g., barbell, plate, kettlebell, dumbbell, medical ball, accessories, etc.) includes an IMU sensor 132. The interactive personal training device 108 is configured to process and analyze the stream of sensor data using trained machine learning algorithms and provide feedback in real time on the user 106 performing the exercise movement. For example, the feedback may include the weight moved in exercise movement pattern, the number of repetitions performed in the exercise movement pattern, the number of sets completed in the exercise movement pattern, the power generated by the exercise movement pattern, etc. In another example, the feedback may include a comparison of the exercise form of the user 106 against conditions of an ideal or correct exercise form predefined for the exercise movement and providing a visual overlay on the interactive display of the interactive personal training device to guide the user 106 to perform the exercise movement correctly. In another example, the feedback may include computation of classical force exerted by the user in the exercise movement and providing an audible and/or visual instruction to the user to increase or decrease force in a direction using motion path guidance on the interactive display of the interactive personal training device. The feedback may be provided visually on the interactive display screen of the interactive personal training device 108, audibly through the speakers of the interactive personal training device 108, or a combination of both. In some implementations, the interactive personal training device 108 may cause one or more light strips on its frame to pulse to provide the user with visual cues (e.g., repetition counting, etc.) representing a feedback. The user 106 may interact with the interactive personal training device 108 using voice commands or gesture-based commands. It should be understood that the sensor(s) 109 on the interactive personal training device 108 may be configured to track movements of multiple people at the same time. Although the example configuration in FIG. 1B is illustrated in the context of tracking physical activity of a user performing exercise movements and providing feedback and recommendations relating to performing the exercise movements, it should be understood that the configuration may apply to other contexts in vertical fields, such as medical diagnosis (e.g., health practitioner reviewing vital signs of a user, volumetric scanning, 3D imaging in medicine, etc.), physical therapy (e.g. physical therapist checking adherence to physio protocols during rehabilitation), and enhancing user experience in commerce including fashion, clothing, and accessories (e.g., virtual shopping with augmented reality try-ons), and body composition scanning in a personal training or coaching capacity.

FIG. 2 is a block diagram illustrating one embodiment of a computing device 200 including a personal training application 110. The computing device 200 may also include a processor 235, a memory 237, a display device 239, a communication unit 241, an optional capture device 245, an input/output device(s) 247, optional sensor(s) 249, and a data storage 243, according to some examples. The components of the computing device 200 are communicatively coupled by a bus 220. In some embodiments, the computing device 200 may be representative of the interactive personal training device 108, the client device 130, the personal training backend server 120, or a combination of the interactive personal training device 108, the client device 130, and the personal training backend server 120. In such embodiments where the computing device 200 is the interactive personal training device 108, the client device 130, or the personal training backend server 120, it should be understood that the interactive personal training device 108 the client device 130, and the personal training backend server 120 may take other forms and include additional or fewer components without departing from the scope of the present disclosure. For example, while not shown, the computing device 200 may include sensors, additional processors, and other physical configurations. Additionally, it should be understood that the computer architecture depicted in FIG. 2 could be applied to other entities of the system 100 with various modifications, including, for example, the servers 140.

The processor 235 may execute software instructions by performing various input/output, logical, and/or mathematical operations. The processor 235 may have various computing architectures to process data signals including, for example, a complex instruction set computer (CISC) architecture, a reduced instruction set computer (RISC) architecture, and/or an architecture implementing a combination of instruction sets. The processor 235 may be physical and/or virtual, and may include a single processing unit or a plurality of processing units and/or cores. In some implementations, the processor 235 may be capable of generating and providing electronic display signals to a display device 239, supporting the display of images, capturing and transmitting images, and performing complex tasks including various types of feature extraction and sampling. In some implementations, the processor 235 may be coupled to the memory 237 via the bus 220 to access data and instructions therefrom and store data therein. The bus 220 may couple the processor 235 to the other components of the computing device 200 including, for example, the memory 237, the communication unit 241, the display device 239, the input/output device(s) 247, the sensor(s) 249, and the data storage 243. In some implementations, the processor 235 may be coupled to a low-power secondary processor (e.g., sensor hub) included on the same integrated circuit or on a separate integrated circuit. This secondary processor may be dedicated to performing low-level computation at low power. For example, the secondary processor may perform sensor fusion, sensor batching, etc. in accordance with the instructions received from the personal training application 110.

The memory 237 may store and provide access to data for the other components of the computing device 200. The memory 237 may be included in a single computing device or distributed among a plurality of computing devices as discussed elsewhere herein. In some implementations, the memory 237 may store instructions and/or data that may be executed by the processor 235. The instructions and/or data may include code for performing the techniques described herein. For example, as depicted in FIG. 2, the memory 237 may store the personal training application 110. The memory 237 is also capable of storing other instructions and data, including, for example, an operating system 107, hardware drivers, other software applications, databases, etc. The memory 237 may be coupled to the bus 220 for communication with the processor 235 and the other components of the computing device 200.

The memory 237 may include one or more non-transitory computer-usable (e.g., readable, writeable) device, a static random access memory (SRAM) device, a dynamic random access memory (DRAM) device, an embedded memory device, a discrete memory device (e.g., a PROM, FPROM, ROM), a hard disk drive, an optical disk drive (CD, DVD, Blu-ray™, etc.) mediums, which can be any tangible apparatus or device that can contain, store, communicate, or transport instructions, data, computer programs, software, code, routines, etc., for processing by or in connection with the processor 235. In some implementations, the memory 237 may include one or more of volatile memory and non-volatile memory. It should be understood that the memory 237 may be a single device or may include multiple types of devices and configurations.

The bus 220 may represent one or more buses including an industry standard architecture (ISA) bus, a peripheral component interconnect (PCI) bus, a universal serial bus (USB), or some other bus providing similar functionality. The bus 220 may include a communication bus for transferring data between components of the computing device 200 or between computing device 200 and other components of the system 100 via the network 105 or portions thereof, a processor mesh, a combination thereof, etc. In some implementations, the personal training application 110 and various other software operating on the computing device 200 (e.g., an operating system 107, device drivers, etc.) may cooperate and communicate via a software communication mechanism implemented in association with the bus 220. The software communication mechanism may include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication may be configured to be secure (e.g., SSH, HTTPS, etc.).

The display device 239 may be any conventional display device, monitor or screen, including but not limited to, a liquid crystal display (LCD), light emitting diode (LED), organic light-emitting diode (OLED) display or any other similarly equipped display device, screen or monitor. The display device 239 represents any device equipped to display user interfaces, electronic images, and data as described herein. In some implementations, the display device 239 may output display in binary (only two different values for pixels), monochrome (multiple shades of one color), or multiple colors and shades. The display device 239 is coupled to the bus 220 for communication with the processor 235 and the other components of the computing device 200. In some implementations, the display device 239 may be a touch-screen display device capable of receiving input from one or more fingers of a user. For example, the display device 239 may be a capacitive touch-screen display device capable of detecting and interpreting multiple points of contact with the display surface. In some implementations, the computing device 200 (e.g., interactive personal training device 108) may include a graphics adapter (not shown) for rendering and outputting the images and data for presentation on display device 239. The graphics adapter (not shown) may be a separate processing device including a separate processor and memory (not shown) or may be integrated with the processor 235 and memory 237.

The input/output (I/O) device(s) 247 may include any standard device for inputting or outputting information and may be coupled to the computing device 200 either directly or through intervening I/O controllers. In some implementations, the input device 247 may include one or more peripheral devices. Non-limiting example I/O devices 247 include a touch screen or any other similarly equipped display device equipped to display user interfaces, electronic images, and data as described herein, a touchpad, a keyboard, a scanner, a stylus, light emitting diode (LED) indicators or strips, an audio reproduction device (e.g., speaker), an audio exciter, a microphone array, a barcode reader, an eye gaze tracker, a sip-and-puff device, and any other I/O components for facilitating communication and/or interaction with users. In some implementations, the functionality of the input/output device 247 and the display device 239 may be integrated, and a user of the computing device 200 (e.g., interactive personal training device 108) may interact with the computing device 200 by contacting a surface of the display device 239 using one or more fingers. For example, the user may interact with an emulated (i.e., virtual or soft) keyboard displayed on the touch-screen display device 239 by using fingers to contact the display in the keyboard regions.

The capture device 245 may be operable to capture an image (e.g., an RGB image, a depth map), a video or data digitally of an object of interest. For example, the capture device 245 may be a high definition (HD) camera, a regular 2D camera, a multi-spectral camera, a structured light 3D camera, a time-of-flight 3D camera, a stereo camera, a standard smartphone camera, a barcode reader, an RFID reader, etc. The capture device 245 is coupled to the bus to provide the images and other processed metadata to the processor 235, the memory 237, or the data storage 243. It should be noted that the capture device 245 is shown in FIG. 2 with dashed lines to indicate it is optional. For example, where the computing device 200 is the personal training backend server 120, the capture device 245 may not be part of the system, where the computing device 200 is the interactive personal training device 108, the capture device 245 may be included and used to provide images, video and other metadata information described below.

The sensor(s) 249 includes any type of sensors suitable for the computing device 200. The sensor(s) 249 are communicatively coupled to the bus 220. In the context of the interactive personal training device 108, the sensor(s) 249 may be configured to collect any type of signal data suitable to determine characteristics of its internal and external environments. Non-limiting examples of the sensor(s) 249 include various optical sensors (CCD, CMOS, 2D, 3D, light detection and ranging (LiDAR), cameras, etc.), audio sensors, motion detection sensors, magnetometer, barometers, altimeters, thermocouples, moisture sensors, infrared (IR) sensors, radar sensors, other photo sensors, gyroscopes, accelerometers, geo-location sensors, orientation sensor, wireless transceivers (e.g., cellular, Wi-Fi™, near-field, etc.), sonar sensors, ultrasonic sensors, touch sensors, proximity sensors, distance sensors, microphones, etc. In some implementations, one or more sensors 249 may include externally facing sensors provided at the front side, rear side, right side, and/or left side of the interactive personal training device 108 in order to capture the environment surrounding the interactive personal training device 108. In some implementations, the sensor(s) 249 may include one or more image sensors (e.g., optical sensors) configured to record images including video images and still images, may record frames of a video stream using any applicable frame rate, and may encode and/or process the video and still images captured using any applicable methods. In some implementations, the image sensor(s) 249 may capture images of surrounding environments within their sensor range. For example, in the context of an interactive personal training device 108, the sensors 249 may capture the environment around the interactive personal training device 108 including people, ambient light (e.g., day or night time), ambient sound, etc. In some implementations, the functionality of the capture device 245 and the sensor(s) 249 may be integrated. It should be noted that the sensor(s) 249 is shown in FIG. 2 with dashed lines to indicate it is optional. For example, where the computing device 200 is the personal training backend server 120, the sensor(s) 249 may not be part of the system, where the computing device 200 is the interactive personal training device 108, the sensor(s) 249 may be included.

The communication unit 241 is hardware for receiving and transmitting data by linking the processor 235 to the network 105 and other processing systems via signal line 104. The communication unit 241 receives data such as requests from the interactive personal training device 108 and transmits the requests to the personal training application 110, for example a request to start a workout session. The communication unit 241 also transmits information including media to the interactive personal training device 108 for display, for example, in response to the request. The communication unit 241 is coupled to the bus 220. In some implementations, the communication unit 241 may include a port for direct physical connection to the interactive personal training device 108 or to another communication channel. For example, the communication unit 241 may include an RJ45 port or similar port for wired communication with the interactive personal training device 108. In other implementations, the communication unit 241 may include a wireless transceiver (not shown) for exchanging data with the interactive personal training device 108 or any other communication channel using one or more wireless communication methods, such as IEEE 802.11, IEEE 802.16, Bluetooth® or another suitable wireless communication method.

In yet other implementations, the communication unit 241 may include a cellular communications transceiver for sending and receiving data over a cellular communications network such as via short messaging service (SMS), multimedia messaging service (MMS), hypertext transfer protocol (HTTP), direct data connection, WAP, e-mail or another suitable type of electronic communication. In still other implementations, the communication unit 241 may include a wired port and a wireless transceiver. The communication unit 241 also provides other conventional connections to the network 105 for distribution of files and/or media objects using standard network protocols such as TCP/IP, HTTP, HTTPS, and SMTP as will be understood to those skilled in the art.

The data storage 243 is a non-transitory memory that stores data for providing the functionality described herein. In some embodiments, the data storage 243 may be coupled to the components 235, 237, 239, 241, 245, 247, and 249 via the bus 220 to receive and provide access to data. In some embodiments, the data storage 243 may store data received from other elements of the system 100 include, for example, the API 136 in servers 140 and/or the personal training applications 110, and may provide data access to these entities. The data storage 243 may store, among other data, user profiles 222, training datasets 224, machine learning models 226, and workout programs 228.

The data storage 243 may be included in the computing device 200 or in another computing device and/or storage system distinct from but coupled to or accessible by the computing device 200. The data storage 243 may include one or more non-transitory computer-readable mediums for storing the data. In some implementations, the data storage 243 may be incorporated with the memory 237 or may be distinct therefrom. The data storage 243 may be a dynamic random access memory (DRAM) device, a static random access memory (SRAM) device, flash memory, or some other memory devices. In some implementations, the data storage 243 may include a database management system (DBMS) operable on the computing device 200. For example, the DBMS could include a structured query language (SQL) DBMS, a NoSQL DMBS, various combinations thereof, etc. In some instances, the DBMS may store data in multi-dimensional tables comprised of rows and columns, and manipulate, e.g., insert, query, update and/or delete, rows of data using programmatic operations. In other implementations, the data storage 243 also may include a non-volatile memory or similar permanent storage device and media including a hard disk drive, a CD-ROM device, a DVD-ROM device, a DVD-RAM device, a DVD-RW device, a flash memory device, or some other mass storage device for storing information on a more permanent basis.

It should be understood that other processors, operating systems, sensors, displays, and physical configurations are possible.

As depicted in FIG. 2, the memory 237 may include the operating system 107 and the personal training application 110.

The operating system 107, stored on memory 237 and configured to be executed by the processor 235, is a component of system software that manages hardware and software resources in the computing device 200. The operating system 107 includes a kernel that controls the execution of the personal training application 110 by managing input/output requests from the personal training application 110. The personal training application 110 requests a service from the kernel of the operating system 107 through system calls. In addition, the operating system 107 may provide scheduling, data management, memory management, communication control and other related services. For example, the operating system 107 is responsible for recognizing input from a touch screen, sending output to a display screen, tracking files on the data storage 243, and controlling peripheral devices (e.g., Bluetooth® headphones, equipment 134 integrated with an IMU sensor 132, etc.). In some implementations, the operating system 107 may be a general-purpose operating system. For example, the operating system 107 may be Microsoft Windows®, Mac OS® or UNIX® based operating system. Or the operating system 107 may be a mobile operating system, such as Android®, iOS® or Tizen™. In other implementations, the operating system 107 may be a special-purpose operating system. The operating system 107 may include other utility software or system software to configure and maintain the computing device 200.

In some implementations, the personal training application 110 may include a personal training engine 202, a data processing engine 204, a machine learning engine 206, a feedback engine 208, a recommendation engine 210, a gamification engine 212, a program enhancement engine 214, and a user interface engine 216. The components 202, 204, 206, 208, 210, 212, 214, and 216 may be communicatively coupled by the bus 220 and/or the processor 235 to one another and/or the other components 237, 239, 241, 243, 245, 247, and 249 of the computing device 200 for cooperation and communication. The components 202, 204, 206, 208, 210, 212, 214, and 216 may each include software and/or logic to provide their respective functionality. In some implementations, the components 202, 204, 206, 208, 210, 212, 214, and 216 may each be implemented using programmable or specialized hardware including a field-programmable gate array (FPGA) or an application-specific integrated circuit (ASIC). In some implementations, the components 202, 204, 206, 208, 210, 212, 214, and 216 may each be implemented using a combination of hardware and software executable by the processor 235. In some implementations, each one of the components 202, 204, 206, 208, 210, 212, 214, and 216 may be sets of instructions stored in the memory 237 and configured to be accessible and executable by the processor 235 to provide their acts and/or functionality. In some implementations, the components 202, 204, 206, 208, 210, 212, 214, and 216 may send and receive data, via the communication unit 241, to and from one or more of the client devices 130, the interactive personal training devices 108, the personal training backend server 120 and third-party servers 140.

The personal training engine 202 may include software and/or logic to provide functionality for creating and managing user profiles 222 and selecting one or more workout programs for users of the interactive personal training device 108 based on the user profiles 222. In some implementations, the personal training engine 202 receives a user profile from a user's social network account with permission from the user. For example, the personal training engine 202 may access an API 136 of a third-party social network server 140 to request a basic user profile to serve as a starter profile. The user profile received from the third-party social network server 140 may include one or more of the user's age, gender, interests, location, and other demographic information. The personal training engine 202 may receive information from other components of the personal training application 110 and use the received information to update the user profile 222 accordingly. For example, the personal training engine 202 may receive information including performance statistics of the user participation in a full body workout session from the feedback engine 208 and update the workout history portion in the user profile 222 using the received information. In another example, the personal training engine 202 may receive achievement badges that the user earned after reaching one or more milestones from the gamification engine 212 and accordingly associate the badges with the user profile 222.

In some implementations, the user profile 222 may include additional information about the user including name, age, gender, height, weight, profile photo, 3D body scan, training preferences (e.g. HIIT, Yoga, barbell powerlifting, etc.), fitness goals (e.g., gain muscle, lose fat, get lean, etc.), fitness level (e.g., beginner, novice, advanced, etc.), fitness trajectory (e.g., losing 0.5% body fat monthly, increasing bicep size by 0.2 centimeters monthly, etc.), workout history (e.g., frequency of exercise, intensity of exercise, total rest time, average time spent in recovery, average time spent in active exercise, average heart rate, total exercise volume, total weight volume, total time under tension, one-repetition maximum, etc.), activities (e.g. personal training sessions, workout program subscriptions, indications of approval, multi-user communication sessions, purchase history, synced wearable fitness devices, synced third-party applications, followers, following, etc.), video and audio of performing exercises, and profile rating and badges (e.g., strength rating, achievement badges, etc.). The personal training engine 202 stores and updates the user profiles 222 in the data storage 243.

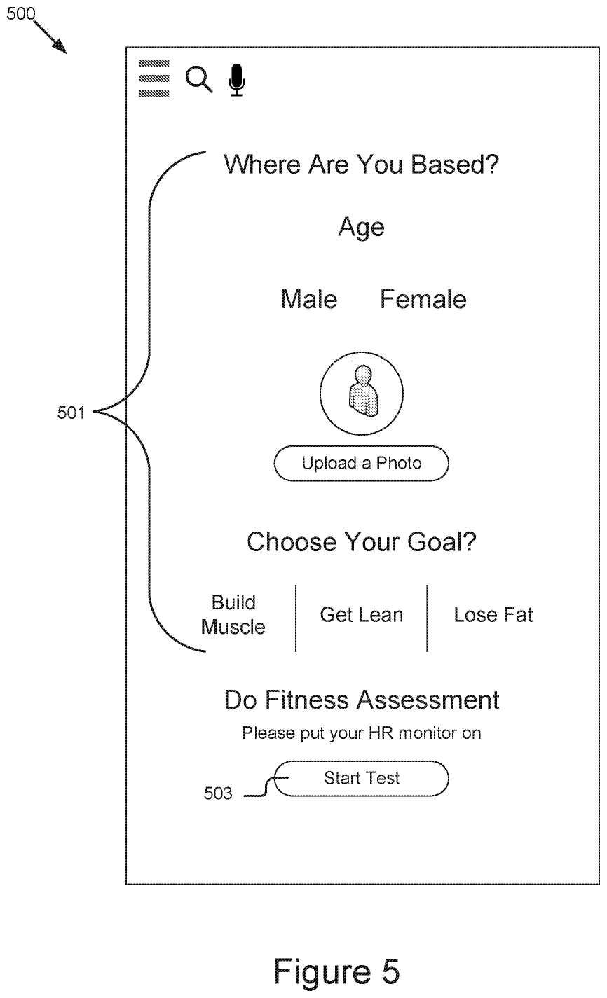

FIG. 5 shows an example graphical representation of a user interface for creating a user profile of a user in association with the interactive personal training device 108. In FIG. 5, the user interface 500 depicts a list 501 of questions that the user may view and provide answers. The answers input by the user are used create a user profile 222. The user interface 500 also includes a prompt for the user to start a fitness assessment test. The user may select the “Start Test” button 503 to undergo an evaluation and a result of this evaluation is added to the user profile 222. The fitness assessment test may include measuring, for example, a heart rate at rest, a target maximum heart rate, muscular strength and endurance, flexibility, body weight, body size, body proportions, etc. The personal training engine 202 cooperates with the feedback engine 208 to assess the initial fitness of the user and updates the profile 222 accordingly. The personal training engine 202 selects one or more workout programs 228 from a library of workout programs based on the user profile 222 of the user. A workout program 228 may define a set of weight equipment-based exercise routines, a set of bodyweight based exercise routines, a set of isometric holds, or a combination thereof. The workout program 228 may be designed for a period of time (e.g., a 4 week full body strength training workout). Example workout programs may include one or more exercise movements based on, cardio, yoga, strength training, weight training, bodyweight exercises, dancing, toning, stretching, martial arts, Pilates, core strengthening, or a combination thereof. A workout program 228 may include an on-demand video stream of an instructor performing the exercise movements for the user to repeat and follow along. A workout program 228 may include a live video stream of an instructor performing the exercise movement in a remote location and allowing for two-way user interaction between the user and the instructor. The personal training engine 202 cooperates with the user interface engine 216 for displaying the selected workout program on the interactive screen of the interactive personal training device 108.

The data processing engine 204 may include software and/or logic to provide functionality for receiving and processing a sensor data stream from a plurality of sensors focused on monitoring the movements, position, activities, and interactions of one or more users of the interactive personal training device 108. The data processing engine 204 receives a first set of sensor data from the sensor(s) 249 of the interactive personal training device 108. For example, the first set of sensor data may include one or more image frames, video, depth map, audio, and other sensor data capturing the user performing an exercise movement in a private or semi-private space. The data processing engine 204 receives a second set of sensor data from an inertial measurement unit (IMIJ) sensor 132 associated with an equipment 134 in use. For example, the second set of sensor data may include physical motion parameters, such as acceleration, velocity, position, orientation, rotation etc. of the equipment 134 used by the user in association with performing the exercise movement. The data processing engine 204 receives a third set of sensor data from sensors available in one or more wearable devices in association with the user performing the exercise movement. For example, the third set of sensor data may include physiological, biochemical, and environmental sensor signals, such as heart rate (pulse), heart rate variability, oxygen level, glucose, blood pressure, temperature, respiration rate, cutaneous water (sweat, salt secretion), saliva biomarkers, calories burned, eye tracking, etc. captured using one or more wearable devices during the user performance of the exercise movement.

In some implementations, the data processing engine 204 receives contextual user data from a variety of third-party APIs 136 for online services 111 outside of an active workout session of a user. Example contextual user data that the data processing engine 204 collects includes, but is not limited to, sleep quality data of the user from a web API of a wearable sleep tracking device, physical activity data of the user from a web API of a fitness tracker device, calories and nutritional intake data from a web API of a calorie counter application, manually inputted gym workout routines, cycling, running, and competition (e.g. marathon, 5 K run, etc.) participation statistics from a web API of a fitness mobile application, a calendar schedule of a user from a web API of a calendar application, social network contacts of a user from a web API of a social networking application, purchase history data from a web API of an e-commerce application, etc. This contextual user data is added to the existing user workout data performed on the interactive personal training device 108 to recommend to the user a workout program based on fatigue levels (e.g., from exercise or poor sleep quality), or nutrient intake (e.g., lack of calories or excess) and exercises the user has performed outside of the interactive personal training device 108 to determine fitness of the user. The data processing engine 204 processes, correlates, integrates, and synchronizes the received sensor data stream and the contextual user data from disparate sources into a consolidated data stream as described herein. In some implementations, the data processing engine 204 time stamps the received sensor data at reception and uses the time stamps to correlate, integrate, and synchronize the received sensor data. For example, the data processing engine 204 synchronizes in time the sensor data received from the IMU sensor 132 on an equipment 134 with an image frame or depth map of the user performing the exercise movement captured by the sensor(s) 249 of the interactive personal training device 108.

In some implementations, the data processing engine 204 in an instance of the personal training application 110a on the interactive personal training device 108 performs preprocessing on the received data at the interactive personal training device 108 to reduce data transmitted over the network 105 to the personal training backend server 120 for analysis. The data processing engine 204 transforms the received data into a corrected, ordered, and simplified form for analysis. By preprocessing the received data at the interactive personal training device 108, the data processing engine 204 enables a low latency streaming of data to the personal training backend server 120 for requesting analysis and receiving feedback on the user performing the exercise movement. In one example, the data processing engine 204 receives image frames of a scene from a depth sensing camera on the interactive personal training device 108, removes non-moving parts in the image frames (e.g., background), and sends the depth information calculated for the foreground object to the personal training backend server 120 for analysis. Other data processing tasks performed by the data processing engine 204 to reduce latency may include one or more of data reduction, data preparation, sampling, sub sampling, smoothing, compression, background subtraction, image cleanup, image segmentation, image rectification, spatial mapping, etc. on the received data. Also, the data processing engine 204 may determine a nearest personal training backend server 120 of a server cluster to send the data for analysis using network ping and associated response times. Other methods for improving latency include direct socket connection, DNS optimization, TCP optimization, adaptive frame rate, routing, etc. The data processing engine 204 sends the processed data stream to other components of the personal training application 110 for analysis and feedback.

In some implementations, the data processing engine 204 curates one or more training datasets 224 based on the data received in association with a plurality of interactive personal training devices 108, the third-party servers 140, and the plurality of client devices 130. The machine learning engine 206 described in detail below uses the training datasets 224 to train the machine learning models. Example training datasets 224 curated by the data processing engine 204 include, but not limited to, a dataset containing a sequence of images or video for a number of users engaged in physical activity synchronized with labeled time-series heart rate over a period of time, a dataset containing a sequence of images or video for a number of users engaged in physical activity synchronized with labeled time-series breathing rate over a period of time, a dataset containing a sequence of images or video for a number of repetitions relating to an labelled exercise movement (e.g., barbell squat) performed by a trainer, a dataset containing images for a number of labelled facial expressions (e.g., strained facial expression), a dataset containing images of a number of labelled equipment (e.g., dumbbell), a dataset containing images of a number of labelled poses (e.g., a downward phase of a squat barbell movement), etc. In some implementations, the data processing engine 204 accesses a publicly available dataset of images that may serve as a training dataset 224. For example, the data processing engine 204 may access a publicly available dataset to use as a training dataset 224 for training a machine learning model for object detection, facial expression detection, etc. In some implementations, the data processing engine 204 may create a crowdsourced training dataset 224. For example, in the instance where a user (e.g., personal trainers, clients, etc.) consents to use of their content for creating a training dataset, the data processing engine 204 receives the video of the user performing one or more unlabeled exercise movements. The data processing engine 204 provides the video to remotely located reviewers that review the video, identify a segment of the video, classify and provide a label for the exercise movement present in the identified segment. The data processing engine 204 stores the curated training datasets 224 in the data storage 243.

The machine learning engine 206 may include software and/or logic to provide functionality for training one or more machine learning models 226 or classifiers using the training datasets created or aggregated by the data processing engine 204. In some implementations, the machine learning engine 206 may be configured to incrementally adapt and train the one or more machine learning models every threshold period of time. For example, the machine learning engine 206 may incrementally train the machine learning models every hour, every day, every week, every month, etc. based on the aggregated dataset. In some implementations, a machine learning model 226 is a neural network model and includes a layer and/or layers of memory units where memory units each have corresponding weights. A variety of neural network models may be utilized including feed forward neural networks, convolutional neural networks, recurrent neural networks, radial basis functions, other neural network models, as well as combinations of several neural networks. Additionally, or alternatively, the machine learning model 226 may represent a variety of other machine learning techniques in addition to neural networks, for example, support vector machines, decision trees, Bayesian networks, random decision forests, k-nearest neighbors, linear regression, least squares, hidden Markov models, other machine learning techniques, and/or combinations of machine learning techniques.

In some implementations, the machine learning engine 206 may train the one or more machine learning models 226 for a variety of machine learning tasks including estimating a pose (e.g., 3D pose (x, y, z) coordinates of keypoints), detecting an object (e.g., barbell, registered user), detecting a weight of the object (e.g., 45 lbs), edge detection (e.g., boundaries of an object or user), recognizing an exercise movement (e.g., dumbbell shoulder press, bodyweight push-up), detecting a repetition of an exercise movement (e.g., a set of 8 repetitions), detecting fatigue in the repetition of the exercise movement, detecting a technique or form of the user in performing the exercise movement within acceptable thresholds, detecting heart rate, detecting breathing rate, detecting blood pressure, detecting facial expression, detecting a risk of injury, etc. In another example, the machine learning engine 206 may train a machine learning model 226 to classify an adherence of an exercise movement performed by a user to predefined conditions for correctly performing the exercise movement. As a further example, the machine learning engine 206 may train a machine learning model 226 to predict the fatigue in a user performing a set of repetitions of an exercise movement. In some implementations, the machine learning model 226 may be trained to perform a single task. In other implementations, the machine learning model 226 may be trained to perform multiple tasks.

The machine learning engine 206 determines a plurality of training instances or samples from the labelled dataset curated by the data processing engine 204. A training instance can include, for example, an instance of a sequence of images depicting an exercise movement classified and labelled as barbell deadlift. The machine learning engine 206 may apply a training instance as input to a machine learning model 226. In some implementations, the machine learning engine 206 may train the machine learning model 226 using any one of at least one of supervised learning (e.g., support vector machines, neural networks, logistic regression, linear regression, stacking, gradient boosting, etc.), unsupervised learning (e.g., clustering, neural networks, singular value decomposition, principal component analysis, etc.), or semi-supervised learning (e.g., generative models, transductive support vector machines, etc.). Additionally, or alternatively, machine learning models 226 in accordance with some implementations may be deep learning networks including recurrent neural networks, convolutional neural networks (CNN), networks that are a combination of multiple networks, etc. The machine learning engine 206 may generate a predicted machine learning model output by applying training input to the machine learning model 226. Additionally, or alternatively, the machine learning engine 206 may compare the predicted machine learning model output with a known labelled output (e.g., classification of a barbell deadlift) from the training instance and, using the comparison, update one or more weights in the machine learning model 226. In some implementations, the machine learning engine 206 may update the one or more weights by backpropagating the difference over the entire machine learning model 226.

In some implementations, the machine learning engine 206 may test a trained machine learning model 226 and update it accordingly. The machine learning engine 206 may partition the labelled dataset obtained from the data processing engine 204 into a testing dataset and a training dataset. The machine learning engine 206 may apply a testing instance from the training dataset as input to the trained machine learning model 226. A predicted output generated by applying a testing instance to the trained machine learning model 226 may be compared with a known output for the testing instance to update an accuracy value (e.g., an accuracy percentage) for the machine learning model 226.

Some examples of training machine learning models for specific tasks relating to tracking user performance of exercise movements are described below. In one example, the machine learning engine 206 trains a Convolutional Neural Network (CNN) and Fast Fourier Transform (FFT) based spectro-temporal neural network model to identify photoplethysmography (PPG) in pulse heavy body parts, such as the face, the neck, biceps, wrists, hands, and ankles. The PPG is used to detect heart rate. The machine learning engine 206 trains the CNN and FFT based spectro-temporal neural network model using a training dataset including segmented images of pulse heavy body parts synchronized with the time-series data of heart rate over a period of time. In another example, the machine learning engine 206 trains a Human Activity Recognition (HAR)-CNN model to identify PPG in torso, arms, and head. The PPG is used to detect breathing rate and breathing intensity. The machine learning engine 206 trains the HAR-CNN model using a training dataset including segmented images of torso, arms, and head synchronized with the time-series data of breathing rate over a period of time. In another example, the machine learning engine 206 trains a Region-based CNN (R-CNN) model to infer 3D pose coordinates for keypoints, such as elbows, knees, wrists, hips, shoulder joints, etc. The machine learning engine 206 trains the R-CNN using a labelled dataset of segmented depth images of keypoints in user poses. In another example, the machine learning engine 206 trains a CNN model for edge detection and identifying boundaries of objects including humans in grayscale image using a labeled dataset of segmented images of objects including humans.

The feedback engine 208 may include software and/or logic to provide functionality for analyzing the processed stream of sensor data from the data processing engine 204 and providing feedback on one or more aspects of the exercise movement performed by the user. For example, the feedback engine 208 performs a real time “form check” on the user performing an exercise movement.

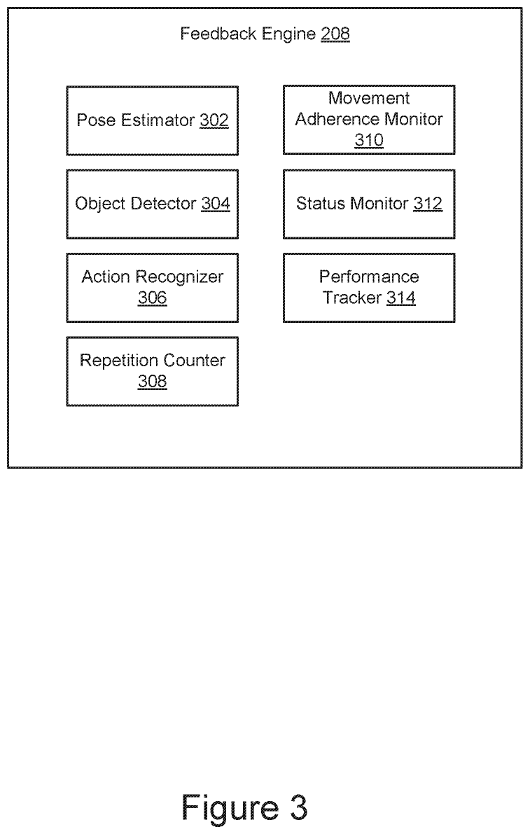

FIG. 3 is a block diagram illustrating an example embodiment of a feedback engine 208. As depicted, the feedback engine 208 may include a pose estimator 302, an object detector 304, an action recognizer 306, a repetition counter 308, a movement adherence monitor 310, a status monitor 312, and a performance tracker 314. Each one of the components 302, 304, 306, 308, 310, 312, and 314 in FIG. 3 may be configured to implement one or more machine learning models 226 trained by the machine learning engine 206 to execute their functionality as described herein. In some implementations, the components 302, 304, 306, 308, 310, 312, and 314 may be interdependent on each other to execute their functionality and therefore organized in such a way to reduce latency. Such an organization may allow for some of the components 302, 304, 306, 308, 310, 312, and 314 to be configured to execute in parallel and some of the components to be configured to execute in sequence. For example, the classification of exercise movement by action recognizer 306 may follow the detection of pose by pose estimator 302 in sequence whereas the detection of object by object detector 304 and detection of pose by pose estimator 302 may execute in parallel. Each one of the components 302, 304, 306, 308, 310, 312, and 314 in FIG. 3 may be configured to transmit their generated result or output to the recommendation engine 210 for generating one or more recommendations to the user.

The pose estimator 302 receives the processed sensor data stream including one or more images from the data processing engine 204 depicting one or more users and estimates the 2D or 3D pose coordinates for each keypoint (e.g., elbows, wrists, joints, knees, etc.). The pose estimator 302 tracks a movement of one or more users in real-world space by predicting the precise location of keypoints associated with the users. For example, the pose estimator 302 receives the RGB image and associated depth map, inputs the received data into a trained convolutional neural network for pose estimation, and generates 3D pose coordinates for one or more keypoints associated with a user. The pose estimator 302 generates a heatmap predicting the probability of the keypoint occurring at each pixel. In some implementations, the pose estimator 302 detects and tracks a static pose in a number of continuous image frames. For example, the pose estimator 302 classifies a pose as a static pose if the user remains in that pose for at least 30 image frames (2 seconds if the image frames are streaming at 15 FPS). The pose estimator 302 determines a position, an angle, a distance, and an orientation of the keypoints based on the estimated pose. For example, the pose estimator 302 determines a distance between the two knees, an angle between a shoulder joint and an elbow, a position of the hip joint relative to the knee, and an orientation of the wrist joint in an articulated pose based on the estimated 3D pose data. The pose estimator 302 determines an initial position, a final position, and a relative position of a joint in a sequence of a threshold number of frames. The pose estimator 302 passes the 3D pose data including the determined position, angle, distance, and orientation of the keypoints to other components 304, 306, 308, 310, 312, and 314 in the feedback engine 208 for further analysis.

In some implementations, the pose estimator 302 analyzes the sensor data including one or more images captured by the interactive personal training device 108 to generate anthropometric measurements including a three-dimensional view of the user's body. For example, the interactive personal training device 108 may receive a sequence of images that capture the details of the user's body in 360 degrees. The pose estimator 302 uses the combination of the sequence of images to generate a 3D visualization (e.g., avatar) of user's body and provides an estimate for body measurements (e.g., arms, thighs, hips, waist, etc.). The pose estimator 302 also determines body size, body shape, and body composition of the user. In some implementations, the pose estimator 302 generates a 3D model of the user (shown in FIG. 4) as a set of connected keypoints and sends the 3D model to the user interface engine 216 for displaying on the interactive screen of the interactive personal training device 108.

The object detector 304 receives the processed sensor data stream including one or more images from the data processing engine 204 and detects one or more objects (e.g., equipment 134) utilized by a user in association with performing an exercise movement. The object detector 304 detects and locates an object in the image using a bounding box encompassing the detected object. For example, the object detector 304 receives the RGB image and associated depth map, inputs the received data into a trained You Only Look Once (YOLO) convolutional neural network for object detection, detects a location of an object (e.g., barbell with weight plates) and an estimated weight of the object. In some implementations, the object detector 304 determines a weight associated with the detected object by performing optical character recognition (OCR) on the detected object. For example, the object detector 304 detects markings designating a weight of a dumbbell in kilograms or pounds. In some implementations, the object detector 304 identifies the type and weight of the weight equipment based on the IMU sensor data associated with the weight equipment. The object detector 304 instructs the user interface engine 216 to display a detection of a weight equipment on the interactive screen of the interactive personal training device 108. For example, as the user picks up a weight equipment equipped with an IMU sensor, the object detector 304 identifies the type as a dumbbell and weight as 25 pounds and the user interface engine 216 displays a text “25 pound Dumbbell Detected.” In some implementations, the object detector 304 performs edge detection for segmenting boundaries of objects including one or more users within the images received over a time frame or period of time. The object detector 304 in cooperation with the action recognizer 306 (described below) uses a trained CNN model on the segmented images of a user extracted using edge detection to classify an exercise movement (e.g., squat movement) of the user. In such implementations, the 3D pose data may be deficient for classifying the exercise movement of the user and thus leading the feedback engine 208 to use edge detection as an alternative option. The action recognizer 306 may use either the estimated 3D pose data or the edge detection data or appropriately weight (e.g., 90% weighting to 3D pose data, 10% weighting to edge detection data) them both for optimal classification of exercise movement. In some implementations, the object detector 304 implements background subtraction to extract the detected object in the foreground for further processing. The object detector 304 determines a spatial distance of the object relative to the user as well as the floor plane or equipment. In some implementations, the object detector 304 detects the face of the user in the one or more images for facial authentication to use the interactive personal training device 108. The object detector 304 may analyze the images to detect a logo on a fitness apparel worn by the user, a style of the fitness apparel, and a fit of the fitness apparel. The object detector 304 passes the object detection data to other components 306, 308, 310, 312, and 314 in FIG. 3 for further analysis.

The action recognizer 306 receives the estimated 3D pose data including the determined position, angle, distance, and orientation of the keypoints from the pose estimator 302 for analyzing the action or exercise movement of the user. In some implementations, the action recognizer 306 sends the 3D pose data to a separate logic defined for each exercise movement. For example, the logic may include a set of if-else conditions to determine whether a detected pose is part of the exercise movement. The action recognizer 306 scans for an action in the received data every threshold number (e.g., 100 to 300) of image frames to determine one or more exercise movements. An exercise movement may have two or more articulated poses that define the exercise movement. For example, a jumping jack is a physical jumping exercise performed by jumping to a first pose with the legs spread wide and hands going overhead, sometimes in a clap, and then returning to a second pose with the feet together and the arms at the sides. The action recognizer 306 determines whether a detected pose in the received 3D pose data matches one of the articulated poses for the exercise movement. The action recognizer 306 further determines whether there is a change in the detected poses from a first articulated pose to a second articulated pose defined for the exercise movement in a threshold number of image frames. Accordingly, the action recognizer 306 identifies the exercise movement based on the above determinations. In the instance of detecting a static pose in the received 3D pose data for a threshold number of frames, the action recognizer 306 determines that the user has stopped performing the exercise movement. For example, a user after performing a set of repetitions of an exercise movement may place their hands on knees in a hunched position to catch their breath. The action recognizer 306 identifies such a static pose as not belonging to any articulated poses for purposes of exercise identification and determines that the user is simply at rest.

The action recognizer 306 receives data including object data from the object detector 304 indicating a detection of an equipment utilized by a user in association with performing the exercise movement. The action recognizer 306 determines a classification of the exercise movement based on the use of the equipment. For example, the action recognizer 306 receives 3D pose data for a squat movement and a bounding box for the object detection performed on the barbell and plates equipment combination and classifies the exercise movement as a barbell squat exercise movement. In some implementations, the action recognizer 306 directs the data including the estimated 3D pose data, the object data, and the one or more image frames into a machine learning model (e.g. Human Activity Recognition (HAR)-convolutional neural network) trained for classifying each exercise movement and identifies a classification of the associated exercise movement. In one example, the HAR convolutional neural network may be trained to classify a single exercise movement. In another example, the HAR convolutional neural network may be trained to classify multiple exercise movements. In some implementations, the action recognizer 306 directs the data including the object data, the edge detection data, and the one or more image frames into a machine learning model (e.g. convolutional neural network) trained for classifying each exercise movement and identifies a classification of the associated exercise movement without using 3D pose data. The action recognizer 306 passes the exercise movement classification results to other components 308, 310, 312, and 314 in FIG. 3 for further analysis.

The repetition counter 308 receives data including the estimated 3D pose data from the pose estimator 302 and the exercise classification result from the action recognizer 306 for determining the consecutive repetitions of an exercise movement. The repetition counter 308 identifies a change in pose over several consecutive image frames of the user from a static pose to one of the articulated poses of the identified exercise movement in the received 3D pose data as the start of the repetition. The repetition counter 308 scans for a change of pose of an identified exercise movement from a first articulated pose to a second articulated pose every threshold number (e.g., 100 to 300) of image frames. The repetition counter 308 counts the detected change in pose (e.g., from a first articulated pose to a second articulated pose) as one repetition of that exercise movement and increases a repetition counter by one. When the repetition counter 308 detects static pose for a threshold number of frames after a series of changing articulated poses for the identified exercise movement, the repetition counter 308 determines that the user has stopped performing the exercise movement, generates a count of the consecutive repetitions detected so far for that exercise movement, and resets the repetition counter. It should be understood that the same HAR convolutional neural network used for recognizing an exercise movement may also be used or implemented by the repetition counter 308 in repetition counting. The repetition counter 308 may instruct the user interface engine 216 to display the repetition counting in real time on the interactive screen of the interactive personal training device 108. The repetition counter 308 may instruct the user interface engine 216 to present the repetition counting via audio on the interactive personal training device 108. The repetition counter 308 may instruct the user interface engine 216 to cause one or more light strips on the frame of the interactive personal training device 108 to pulse for repetition counting. In some implementations, the repetition counter 308 receives edge detection data including segmented images of the user actions over a threshold period of time and process the received data for identifying waveform oscillations in the signal stream of images. An oscillation may be present when the exercise movement is repeated. The repetition counter 308 determines a repetition of the exercise movement using the oscillations identified in the signal stream of images.

The movement adherence monitor 310 receives data including the estimated 3D pose data from the pose estimator 302, the object data from the object detector 304, the exercise classification result from the action recognizer 306, and the consecutive repetitions of the exercise movement from the repetition counter 308 for determining whether the user performance of one or more repetitions of the exercise movement adhere to predefined conditions or thresholds for correctly performing the exercise movement. A personal trainer or a professional may define conditions for a proper form or technique associated with performing an exercise movement. In some implementations, the movement adherence monitor 310 may use a CNN model on a dataset containing repetitions of an exercise movement to determine the conditions for a proper form. The form may be defined as a specific way of performing the exercise movement to avoid injury, maximize benefit of exercise movement, and increase strength. To this end, the personal trainer may define the position, angle, distance, and orientation of keypoints, such as joints, wrists, ankles, elbows, knees, back, head, shoulders, etc. in the recognized way of performing a repetition of the exercise movement. In some implementations, the movement adherence monitor 310 compares whether the user performance of the exercise movement in view of body mechanics associated with correctly performing the exercise movement falls within acceptable range or threshold for human joint positions and movements. In some implementations, the movement adherence monitor 310 uses a machine learning model, such as a convolutional neural network trained on a large set of ideal or correct repetitions of an exercise movement to determine a score or a quality of the exercise movement performed by the user based at least on the estimated 3D pose data and the consecutive repetitions of the exercise movement. For example, the score (e.g., 85%) may indicate the adherence to predefined conditions for correctly performing the exercise movement. The movement adherence monitor 310 sends the score determined for the exercise movement to the recommendation engine 210 to generate one or more recommendations for the user to improve the score.

Additionally, the movement adherence monitor 310 receives data including processed sensor data relating to an IMU sensor 132 on the equipment 134 according to some implementations. The movement adherence monitor 310 determines equipment related data including acceleration, spatial location, orientation, and duration of movement of the equipment 134 in association with the user performing the exercise movement. The movement adherence monitor 310 determines an actual motion path of the equipment 134 relative to the user based on the acceleration, the spatial location, the orientation, and duration of movement of the equipment 134. The movement adherence monitor 310 determines a correct motion path using the predefined conditions for the recognized way of performing the exercise movement. The movement adherence monitor 310 compares the actual motion path and the correct motion path to determine a percentage difference to an ideal or correct movement. If the percentage difference meets and/or exceeds a threshold (e.g., 5% and above), the movement adherence monitor 310 instructs the user interface engine 216 to present an overlay of the correct motion path on the display of the interactive personal training device 108 to guide the exercise movement of the user toward the correct motion path. If the percentage difference is within threshold (e.g., between 1% and 5% variability), the movement adherence monitor 310 sends instructions to the user interface engine 216 to present the percentage difference to ideal movement on the display of the interactive personal training device 108. In other implementations, the movement adherence monitor 310 may instruct the user interface engine 216 to display a movement range meter indicating how close the user is performing an exercise movement according to conditions predefined for the exercise movement. Additionally, the movement adherence monitor 310 may instruct the user interface engine 216 to display optimal acceleration and deceleration curve in the correct motion path for performing a repetition of the exercise movement.

The status monitor 312 receives the processed sensor data including the images and estimated 3D pose data from the pose estimator 302 for determining and tracking vital signs and health status of the user during and after the exercise movement. For example, the status monitor 312 uses multispectral imaging technique on data including the received images to identify small changes in the RGB (Red, Green, and Blue) spectrum of the user's face and determine remote heart rate readings based on photoplethysmography (PPG). The status monitor 312 stabilizes the movements in the received images by applying smoothing before determining the remote heart rate readings. In some implementations, the status monitor 312 uses trained machine learning classifiers to determine the health status of the user. For example, the status monitor 312 inputs the RGB sequential images, depth map, and 3D pose data into a trained convolutional neural network for determining one or more of a heart rate, heart rate variability, breathing rate, breathing intensity, blood pressure, facial expression, sweat, etc. In some implementations, the status monitor 312 also receives data relating to the measurements recorded by the wearable devices and uses them to supplement the tracking of vital signs and health status. For example, the status monitor 312 determines an average heart rate based on the heart rate detected using a trained convolutional neural network and a heart rate measured by a heart rate monitor device worn by the user while performing the exercise movement. In some implementations, the status monitor 312 may instruct the user interface engine 216 to display the tracked vital signs and health status on the interactive personal training device 108 in real time as feedback. For example, the status monitor 312 may instruct the user interface engine 216 to display the user's heart rate on the interactive screen of the interactive personal training device 108.

The performance tracker 314 receives the output generated by other components 302, 304, 306, 308, 310, 312 and 314 of the feedback engine 208 in addition to the processed sensor data stream from the data processing engine 204. The performance tracker 314 determines performance statistics and metrics associated with user workout. The performance tracker 314 enables filtering of the performance statistics and metrics by time range, comparing of the performance statistics and metrics from two or more time ranges, and comparing the performance statistics and metrics with other users. The performance tracker 314 instructs the user interface engine 216 to display the performance statistics and metrics on the interactive screen of the interactive personal training device 108. In one example, the performance tracker 314 receives the estimated 3D pose data, the object detection data, the exercise movement classification data, and duration of the exercise movement to determine the power generated by the exercise movement. In another example, the performance tracker 314 receives information on the amount of weight lifted and the number of repetitions in the exercise movement to determine a total weight volume. In another example, the performance tracker 314 receives the estimated 3D pose data, the number of repetitions, equipment related IMU sensor data, and duration of the exercise movement to determine time-under-tension. In another example, the performance tracker 314 determines the amount of calories burned using the metrics output, such as time-under-tension, power generated, total weight volume, and the number of repetitions. In another example, the performance tracker 314 determines a recovery rate indicating how fast a user recovers from a set or workout session using the metrics output, such as power generated, time-under-tension, total weight volume, duration of activity, heart rate, detected facial expression, breathing intensity, and breathing rate.

Other examples of performance metrics and statistics include, but not limited to, total rest time, energy expenditure, current and average heart rate, historical workout data compared with current workout session, completed repetitions in an ongoing workout set, completed sets in an ongoing exercise movement, incomplete repetition, etc. The performance tracker 314 derives total exercise volume from individual workout sessions over a length of time, such as daily, weekly, monthly, and annually. The performance tracker 314 determines total time under tension expressed in seconds or milliseconds using active movement time and bodyweight or equipment weight. The performance tracker 314 determines a total time of exercise expressed in minutes as total length of workout not spent in recovery or rest. The performance tracker 314 determines total rest time from time spent in idle position, such as standing, lying down, hunched over, or sitting. The performance tracker 314 determines total weight volume by multiplying bodyweight by number of repetitions for exercises without weights and multiplying equipment weight by number of repetitions for exercises with weights. As a secondary metric, the performance tracker 314 derives work capacity by dividing the total weight volume by total time of exercise. The performance tracker 314 cooperates with the personal training engine 202 to store the performance statistics and metrics in association with the user profile 222 in the data storage 243. In some implementations, the performance tracker 314 retrieves historical user performance of a workout similar to a current workout of the user and generates a summary comparing the historical performance metrics with the current workout as a percentage to indicate user progress.

Referring to FIG. 4, the example graphical representation illustrates a 3D model of a user as a set of connected keypoints and associated analysis results generated by the components 302, 304, 306, 308, 310, 312, and 314 of the feedback engine 208.

Referring back to FIG. 2, the recommendation engine 210 may include software and/or logic to provide functionality for generating one or more recommendations in real time based on data including user performance. The recommendation engine 210 receives one or more of the 3D pose data, the exercise movements performed, the quality of exercise movements, the repetitions of the exercise movements, the vital signs and health status signals, performance data, object detection data, user profile, and other analyzed user data from the feedback engine 208 and the data processing engine 204 to compare a pattern of the user's workout with an aggregate user dataset (collected from multiple users) to identify a community of users with common characteristics. For example, the common characteristics may include an age group, gender, weight, height, fitness preference, similar performance and workout patterns. In one example, this community of users may be identified by comparing the estimated 3D pose data of users performing the exercise movements over a period of time. The recommendation engine 210 uses both individual user data and aggregate user data to analyze the individual user's workout pattern, user preferences, compare the user's performance data with other similarly performing users, and generate recommendations for users (e.g., novice, pro-athletes, etc.) in real time.

In some implementations, the recommendation engine 210 processes the aggregate user dataset to tag a number of action sequences where multiple users in the identified community of users perform a plurality of repetitions of a specific exercise movement (e.g., barbell squat). The recommendation engine 210 uses the tagged sequences from the aggregate user dataset to train a machine learning model (e.g., CNN) to identify or predict a level of fatigue in the exercise movement. A fatigue in exercise movement may be apparent from a user's inability to move a weight equipment or their own bodyweight at a similar speed, consistency, and steadiness over the several repetitions of the exercise movement. The recommendation engine 210 processes the sequence of user's repetitions of performing the exercise movement using the trained machine learning model to classify the user's experience with the exercise movement and determine the user's current state of fatigue and ability to continue performing the exercise movement. In some implementations, the recommendation engine may track fatigue by muscle group. Additionally, the recommendation engine 210 uses contextual user data including sleep quality data, nutritional intake data, and manually tracked workouts outside the context of the interactive personal training device 108 to predict a level of user fatigue.

The recommendation engine 210 generates on-the-fly recommendation to modify or alter the user exercise workout based on the state or level of fatigue of the user. For example, the recommendation engine 210 may recommend to the user to push for As Many Repetitions As Possible (AMRAP) in the last set of an exercise movement if the level of fatigue of the user is low. In another example, the recommendation engine 210 may recommend to the user to reduce the number of repetitions from 10 to five on a set of exercise movements if the level of fatigue of the user is high. In another example, the recommendation engine 210 may recommend to the user to increase weight on the weight equipment by 10 pounds if the level of fatigue of the user is low. In yet another example, the recommendation engine 210 may recommend to the user to decrease weight on the weight equipment by 20 pounds if the level of fatigue of the user is high. The recommendation engine 210 may also take into account any personally set objectives, a last occurrence of a workout session, number of repetitions of one or more exercise movements, heart rate, breathing rate, facial expression, weight volume, etc. to generate a recommendation to modify the user exercise workout to prevent a risk of injury. For example, the recommendation engine 210 uses Heart Rate Variability (PPG-HRV) in conjunction with exercise analysis to recommend a change in exercise patterns (e.g. if PPG-HRV is poor, recommend a lighter workout). In some implementations, the recommendation engine 210 instructs the user interface engine 216 to display the recommendation on the interactive screen of the interactive personal training device 108 after the user completes a set of repetitions or at the end of the workout session. Example recommendations may include a set amount of weight to pull or push, a number of repetitions to perform (e.g., push for one more rep), a set amount of weight to increase on an exercise movement (e.g., add 10 pound plate for barbell deadlift), a set amount of weight to decrease on an exercise movement (e.g., remove 20 pound plate for barbell squat), a change in an order of exercise movements, change a cadence of the repetition, increase a speed of an exercise movement, decrease a speed of an exercise movement (e.g. reduce the duration of eccentric movement by 1 second to achieve 10% strength gain over 2 weeks), an alternative exercise movement (e.g., do goblet squat instead) to achieve a similar exercise objective, a next exercise movement, a stretching mobility exercise to improve a range of motion, etc.

In some implementations, the recommendation engine 210 receives actual motion path in association with a user using an equipment 134 performing an exercise movement from the movement adherence monitor 310 in the feedback engine 208. The recommendation engine 210 determines the direction of force used by the user in performing the exercise movement based on the actual motion path. If the percentage difference between the actual motion path and the correct motion path is not within a threshold limit, the recommendation engine 210 instructs the user interface engine 216 to generate an alert on the interactive screen of the interactive personal training device 108 informing the user to decrease force in the direction of the actual motion path to avoid injury. In some implementations, the recommendation engine 210 instructs the user interface engine 216 to generate an overlay over the reflected image of the user performing the exercise movement to show what part of their body is active in the exercise movement. For example, the user may be shown with their thigh region highlighted by an overlay in the interactive personal training device 108 to indicate that their quadriceps muscle group are active during a squat exercise movement. By viewing this overlay, the user may understand which part of their body must feel being worked in the performance of a particular exercise movement. In some implementations, the recommendation engine 210 instructs the user interface engine 216 to generate an overlay of the user's prior performance of an exercise movement over the reflected image of the user performing the same exercise movement to show the user their past repetition and speed from a previous workout session. For example, the user may remember how the exercise movement was previously performed by viewing an overlay of their prior performance on the interactive screen of the interactive personal training device 108. In another example, the recommendation engine 210 may overlay a personal trainer performing the exercise movement on the interactive screen of the interactive personal training device 108. The recommendation engine 210 may determine a score for the repetitions of the exercise movement and show comparative progress of the user in performing the exercise movement from prior workouts.

In some implementations, the recommendation engine 210 receives the user profile of a user, analyzes the profile of the user, and generates one or more recommendations based on the user profile. The recommendation engine 210 recommends an optimal workout based on the historical performance statistics and workout pattern in the user profile. For example, the recommendation engine 210 instructs the user interface engine 216 to generate a workout recommendation tile on the interactive screen of the interactive personal training device 108 based on profile attributes, such as a last time the user exercised a particular muscle group, an intensity level (e.g., heart rate) of a typical workout session, a length of the typical workout session, the number of days since the last workout session, an age of the user, sleep quality data, etc. The recommendation engine 210 uses user profiles of other similarly performing users in generating workout recommendations for a target user. For example, the recommendation engine 210 analyzes the user profiles of similarly performing users who have done similar workouts, their ratings for the workouts, and their overall work capacity progress similar to the target user to generate recommendations.

In some implementations, the recommendation engine 210 recommends fitness-related items for user purchase based on the user profile. For example, the recommendation engine 210 determines user preference for a fitness apparel based on the detected logo on their clothing and recommends similar or different fitness apparel for the user to purchase. The recommendation engine 210 may identify the fit and style of the fitness apparel typically worn by the user and accordingly generate purchase recommendations. In another example, the recommendation engine 210 may recommend to the user the fitness apparel worn by a personal trainer to whom the user subscribes for daily workouts. The recommendation engine 210 may instruct the user interface engine 216 to generate an augmented reality overlay of the selected fitness apparel over the reflected image of the user to enable the user to virtually try on the purchase recommendations before purchasing. The recommendation engine 210 cooperates with a web API of an e-commerce application on the third-party server 140 to provide for frictionless purchasing of items via the interactive personal training device 108.

In some implementations, the recommendation engine 210 recommends to the user a profile of a personal trainer or another user to subscribe and follow. The recommendation engine 210 determines workout history, training preferences, fitness goals, etc. of a user based on their user profile and recommends other users who may have more expertise and share similar interests or fitness goals. For example, the recommendation engine 210 generates a list of top 10 users who are strength training enthusiasts matching the interests of a target user on the platform. Users can determine what these successful users have done to achieve their fitness goals at an extremely granular level. The user may also follow other users and personal trainers by subscribing to the workout feed on their user profiles. In addition to the feed that provides comments, instructions, tips, workout summaries and history, the user may see what workouts they are doing and then perform those same workouts with the idea of modelling their favorite users.

The gamification engine 212 may include software and/or logic to provide functionality for managing, personalizing, and gamifying the user experience for exercise workout. The gamification engine 212 receives user performance data, user workout patterns, user competency level, user fitness goals, and user preferences from other components of the personal training application 110 and unlocks one or more workout programs (e.g., live instruction and on-demand classes), peer-to-peer challenges, and new personal trainers. For example, the gamification engine 212 rewards the user by unlocking a new workout program more challenging than a previous workout program that the user has successfully completed. This helps safeguard the user from trying out challenging or advanced workouts very early in their fitness journey and losing motivation to continue their workout. The gamification engine 212 determines a difficulty associated with a workout program based at least on heart rate, lean muscle mass, body fat percentage, average recovery time, exercise intensity, strength progression, work capacity, etc. required to complete the workout program in a given amount of time. Users gain access to new unlocked workout programs based on user performance from doing every repetition and moving appropriate weights in those repetitions for exercise movements in prior workout programs.

The gamification engine 212 instructs the user interface engine 216 to stream the on-demand and live instruction classes for the user on the interactive screen of the interactive personal training device 108. The user may see the instructor or trainer, perform the exercise movement via the streaming video and follow their instruction. The instructor may commend the user on a job well done in a live class based on user performance statistics and metrics. The gamification engine 212 may configure a multiuser communication session (e.g., video chat, text chat, etc.) for a user to interact with the instructor or other users attending the live class via their smartphone device or interactive personal training device 108. In some implementations, the gamification engine 212 manages booking of workout programs and personal trainers for a user. For example, the gamification engine 212 receives a user selection of an upcoming workout class or an unlocked and available personal trainer for a one-on-one training session on the interactive screen of the interactive personal training device 108, books the selected option, and sends a calendar invite to the user's digital calendar. In some implementations, the gamification engine 212 configures two or more interactive personal training devices 108 at remote locations for a partner-based workout session using end-to-end live video streaming and voice chat. For example, a partner-based workout session allows a first user to perform one set of exercise movements and a second user (e.g., a partner of the first user) to perform the next set of exercise movements while the first user rests and vice versa.

The gamification engine 212 enables a user to subscribe to a personal trainer, coach, or a pro-athlete for obtaining individualized coaching and personal training via the interactive personal training device 108. For example, the personal trainer, coach, or pro-athlete may create a subscription channel of live and on-demand fitness streaming videos on the platform and a user may subscribe to the channel on the interactive personal training device 108. Through the channel, the personal trainer, coach, or pro-athlete may offer free group classes and/or fee-based one-on-one personal training to other users. The channel may offer program workouts curated by the personal trainer, coach, or pro-athlete. The program workouts may contain video of exercise movements performed by personal trainer, coach, or pro-athlete for the subscribing user to follow and receive feedback in real time on the interactive personal training device 108. In some implementations, the gamification engine 212 facilitates for the creator of the program workout to review workout history including a video of the subscribing user performing the exercise movements and performance statistics and metrics of the user. They may critique the user's form and provide proprietary tips and suggestions to the user to improve their performance.

The gamification engine 212 allows users to earn achievement badges by completing a milestone that qualify them as competent. The gamification engine 212 monitors the user performance data on a regular basis and suggests new achievement badges to unlock or presents the achievement badges to the user to associate with their user profile in the community of users. For example, the achievement badges may include one or more of a badge for completing a threshold number of workout sessions consistently, a badge for reaching a power level ‘n’ in strength training, a badge for completing a fitness challenge, a badge for unlocking access to a more difficult workout session, a badge for unlocking and winning a peer competition with other users of similar competence and performance levels, a badge for unlocking access to a particular personal trainer, etc. In some implementations, the gamification engine 212 allows the users to share their data including badges, results, workout statistics and performance metrics with a social network of user's choice. The gamification engine 212 receives likes, comments, and other user interactions on the shared user data and displays them in association with the user profile. The gamification engine 212 cooperates with the pose estimator 302 to generate a 3D body scan for accurately visualizing the body transformations of users including body rotations over time and enables sharing of the body transformations on a social network.

In some implementations, the gamification engine 212 may generate a live leaderboard allowing users to view how they rank against their peers on a plurality of performance metrics. For example, the leaderboard may present the user's ranking against friends, regional communities, and/or the entire community of users. The ranking of users shown on the leaderboard can be sorted by a plurality of performance metrics. The plurality of performance metrics may include, for example, overall fitness (strength, endurance, total volume, volume under tension, power, etc.), overall strength, overall endurance, most number of workouts, age, gender, age groups, similar performance, number of peer-to-peer challenges won, champions, attendance in most number of classes, open challenges, etc. In some implementations, the gamification engine 212 may create a matchup between two users on the leaderboard or from personal contacts on the platform to compete on a challenge based on their user profiles. For example, users may be matched up based on similar performance metrics and workout history included in the user profiles. A fitness category may be selected on which to challenge and compete including, for example, a time-based fitness challenge, a strength challenge, an exercise or weight volume challenge, endurance challenge, etc. In some implementations, the challenge may be public or visible only to the participants.

In some implementations, the gamification engine 212 may facilitate users to “level up” a virtual self or avatar based on their preferred physical body representation by following or performing exercise programs or routines, completing workout challenges, and/or collecting achievement badges. For example, the virtual self may be user selectable and be used to show a progress and a current state of the user in their fitness journey. In another example, the virtual self may be a 3D scan representation of the user's actual full-body appearance obtained on a periodic basis by the interactive personal training device 108. The virtual self may include or represent a real time or close to real time information about the user's fitness activity, such as progress level, current state of fitness, personal bests, achievements, workout streaks, etc. In some implementations, the gamification engine 212 presents, for display on the interactive screen, the virtual self after a current session of the workout is done by the user. In some implementations, the gamification engine 212 presents the virtual self during the current session of the workout. The avatar including the real time information about the user's fitness activity may be shared with or made visible to other users (e.g., friends) via the interactive personal training device 108 or via a social media application.

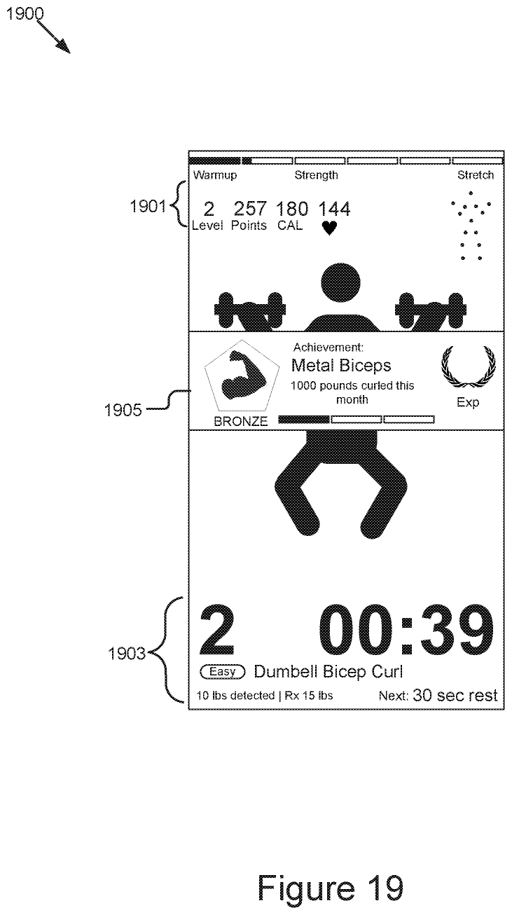

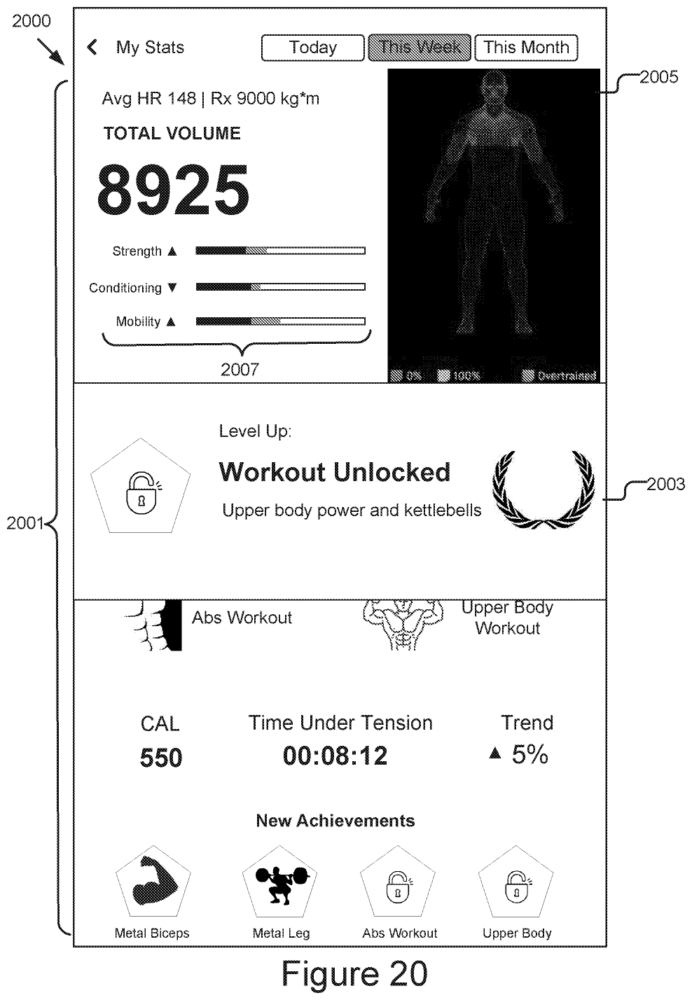

FIG. 19 shows an example graphical representation illustrating user interface 1900 for displaying real time feedback on the interactive personal training device 108. The user interface 1900 depicts an interactive screen on the interactive personal training device 108 displaying real time feedback 1901 and 1903. The feedback 1901 includes a heart rate in beats per minute, calories burned, points earned from completing or performing an exercise routine, and a current level of user's fitness progression. The feedback 1903 includes identification of a type of exercise movement being performed, an amount of time spent or left in the working set, a detected amount of weight being moved in the exercise movement, an active count of number of repetitions completed, an active count of number of sets completed, and an amount of resting time to be had after completion of the working sets. The user interface 1900 depicts a notification 1905 indicating that the user has earned an achievement badge for arm curling 1000 pounds in a month. FIG. 20 shows an example graphical representation illustrating a user interface 2000 for displaying statistics relating to the user completion of an exercise workout session. The user interface 2000 depicts a display of statistics 2001 on the interactive screen of the interactive personal training device 108. For example, the statistics 2001 describe information about total volume, average heart rate, time under tension, number of calories burned in the workout session, a trend in the progress of the user's fitness level, etc. The user accumulates a number of fitness points based on completing all the requirements (e.g., recommended workout sessions, challenges, etc.) of a particular fitness level. When the number of fitness points satisfy or meet a threshold for the next fitness level, the gamification engine 212 unlocks subsequent workout programs and levels up the virtual self of the user. The user interface 2000 depicts a notification 2003 of a workout program being unlocked and/or level up in fitness level on the interactive screen of the interactive personal training device 108.

In some implementations, the gamification engine 212 receives, as input, one or more of the calorie and nutrient intake data, activity data (e.g., hours of inactive state, hours of sleep in a day, hiking, running, number of steps walked, number of floors climbed, etc.), heart rate, heart rate variability, number of breaths per minute, body temperature, blood pressure, other vital signs and health status signals, volume of weight moved during exercise movements, etc. from the feedback engine 208 and the data processing engine 204 for implementing its functionality described herein. The gamification engine 212 in cooperation with the recommendation engine 210 analyzes the collected input data using one or more machine learning models and generates a prediction of a next set of actions for the user to perform. For example, the gamification engine 212 generates a recommendation of one or more adaptive workout programs for the user to perform in their next workout session based on one or more of the above mentioned inputs and the user's performance in the prior and/or ongoing workout sessions. The gamification engine 212 uses a plurality of trained machine learning algorithms to personalize and recommend a next set of workout programs for users as well as how to modify workouts or exercise for those new workout programs. For example, the gamification engine 212 uses one or more trained machine learning algorithms in a weighted manner or in a neural network to generate predictions of next actions. The gamification engine 212 improves with predictions of workout recommendations over time. The workout program recommendations aim to drive up overall user engagement, volume of exercises performed, and user fitness level measured in one or more fitness areas, such as conditioning, strength, and mobility. In some implementations, the gamification engine 212 generates a set of workout recommendations for a user to maximize and/or balance development in one or more fitness areas of strength, mobility, and conditioning. For example, the gamification engine 212 uses a fitness goal (e.g., triathlon training, CrossFit training, etc.) of a user to maximize and/or balance development in one or more of strength, mobility, and conditioning over a predetermined period of time.

In some implementations, the gamification engine 212 receives data including one or more of the 3D pose data, the exercise movements performed, the quality of exercise movements, a number of warm up exercises, the number of repetitions of the exercise movements, the vital signs and health status signals, exercise performance data, object detection data (e.g., weight of the equipment 134 received from an associated IMU sensor 132 built into or attached to the equipment 134), and other user data (e.g., sleep, nutrition, activity, etc.) from the feedback engine 208 and the data processing engine 204 for a workout session completed by the user. The gamification engine 212 processes the received data using one or more machine learning algorithms to identify which of the body parts (e.g., chest, biceps, quadriceps, etc.) were trained and their degree of training (e.g., undertrained, overtrained, optimally trained, etc.) based on time under tension and recommend a set of next workout routines or program to maximize and/or balance development in one or more fitness areas of strength, mobility, and conditioning. For example, the gamification engine 212 uses a neural network to track a user's state of fatigue while working out one or more muscle groups throughout the workout session. The gamification engine 212 instructs the user interface engine 216 to generate an overlay of the virtual self on the interactive screen of the interactive personal training device 108. The gamification engine 212 generates a heat map to highlight parts of the physical body on the virtual self in response to user's performance of a set of exercise routines in a workout session. For example, the gamification engine 212 translates the exercise routines performed by the user to a view of the heat map that highlights body parts or muscle groups (e.g., biceps, quadriceps, shoulders, chest, abs, traps, hamstrings, etc.) which were overtrained, undertrained, or optimally trained. In one example, this determination may be based on an amount of fatigue experienced by one or more of the muscle groups based on their time under tension. In FIG. 20, the user interface 2000 includes a depiction of the virtual self 2005 of the user highlighting the body parts that were trained during a workout session. The user interface 2000 includes a depiction of progress bar 2007 in areas of fitness, such as strength, conditioning, and mobility over different periods of time, such as month, week, and day. The gamification engine 212 generates the next set of workout recommendations for the user in order to fill up the progress bar to full or close to full in one or more fitness areas based on the overall current fitness of the user, the fitness goal of the user, and a predetermined period of time (e.g., eight week fitness program). The gamification engine 212 generates a recommendation for a set of workout routines that include adaptive training changes for the next workout session of the user. For example, the recommended set of workout routines may be a group workout class (e.g. Group yoga) or a custom generated workout for the user. The recommendations for user workouts may also be influenced by a social connection of the user, such as a friend. For example, the gamification engine 212 may recommend a workout to the user that was done by their friend who trains using their own interactive personal training device 108.

In some implementations, the gamification engine 212 generates a recommendation for the user to connect with another user, such as a personal trainer who may review the user's progress and adherence to workout regimen to make adaptive training changes. The gamification engine 212 determines a trend in the user's adherence to work out schedule for recommending a personal trainer. For example, the gamification engine 212 determines a trend with the user missing about 50% of the scheduled or booked workout sessions in association with the interactive personal training device 108 and based on other data collected (e.g., activity tracker data) about the user outside the context of the interactive personal training device 108. The gamification engine 212 recommends a personal trainer or coach to the user for improving accountability, user engagement, and workout experience. Such a personal trainer may be an experienced professional providing training to individual users of other interactive personal training devices 108. The personal trainer may review the workout history of the user and recommend a personalized workout plan for the user.

In some implementations, the gamification engine 212 in cooperation with the feedback engine 208 analyzes the user workout session to track one or more of exercise repetitions, weight equipment usage, adherence to proper form, and user progress in the workout program. The gamification engine 212 generates a compressed timeline of the user's workout over a period of time. For example, the gamification engine 212 captures video of the user's workout in a recent workout session via the interactive personal training device 108, generates a condensed video of the user's workout that focuses on the core or primary exercise movements performed by the user, and adds metadata, such as repetition count, detected weights usage, adherence score for proper form, progress, etc. to the condensed video. The gamification engine 212 provides the personal trainer on-demand access to the condensed video for review and feedback. This benefits the personal trainer because they do not have to review the user's workout live in real time, review the entire duration of their workout, or track the user performance in the workout. The metadata added to the condensed video provides context relating to the user performance in the workout. The personal trainer may asynchronously review the user's workout in a compressed form (e.g., a 60 minute video compressed to 5 minute highlight video) at their own leisure and provide feedback. This feature also reduces the workload for the personal trainer by enabling the personal trainer to manage a large number of clients efficiently and personalize workout recommendations for each client. In one example, the personal trainer or coach may commend or encourage the user on their form or technique after review of their workout. In another example, the personal trainer or coach may recommend a change in diet (e.g., increase protein intake to 140 grams per day) for the user, a set of new workout routines or modifications to existing workout routines, and/or a purchase of an exercise equipment 134 for performing a set of workout routines. The recommendations provided by the personal trainer may be during a one-on-one session on the interactive personal training device 108 or received by the user when they next use the interactive personal training device 108. In some implementations, the gamification engine 212 surfaces the recommendations on a client device 130, such as a smartphone, fitness tracker, tablet, etc. In some implementations, the gamification engine 212 generates a recommendation for the user to join an online community of other users of interactive personal training device 108 or friends to stay accountable in adhering to their workout program. For example, when the user consistently adheres to a workout program exercising with the interactive personal training device 108 for a week, the gamification engine 212 shares this success streak of the user with the online community or friends of the user. The gamification engine 212 facilitates the sharing of workout related information, such as a video, avatar, score, statistics, rewards, progress, level-ups, achievement badges, etc. via a social media application for receiving social reinforcement in the form of indications of acknowledgment (e.g., likes, comments, shares, etc.), feedback, support, and recommendations from a social circle that help with motivating the user. For example, the user may record and share a short-form video of an exercise repetition or an exercise movement that they consider to be their personal record to their social circle via the interactive personal training device 108.

Examples of machine learning algorithms comprising the neural network used by the gamification engine 212 may include, but are not limited to, an overtraining algorithm, a mobility optimization algorithm, a strength optimization algorithm, a conditioning optimization algorithm, a warmup optimization algorithm, an antagonist exercise recommendation algorithm, etc. Examples of adaptive training changes recommended for the next workout session may include, but are not limited to, increase or decrease in volume or weight, type of workout to target muscle groups undertrained, automatic reduction in sets and/or repetitions of exercise movements for muscle groups overtrained, temporary removal of exercises for muscle groups overtrained, type of workout targeting antagonist muscles, increase in mobility or recovery based exercises and stretches for muscle groups overtrained, recently trained, or for a future target fitness goal.

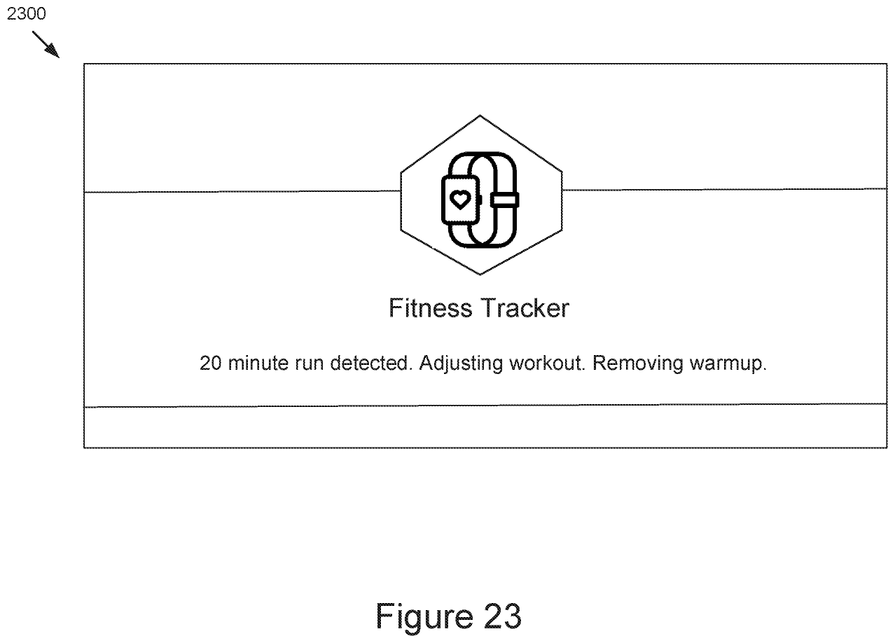

In some implementations, the gamification engine 212 receives contextual user data before and/or after a workout session, such as physical activity data of the user from a web API of a fitness tracker device. The gamification engine 212 processes the data using a neural network to recommend a workout program to the user. For example, if the contextual user data is indicating that the user has been sedentary for most of the day, the workout recommendation for the user would lean toward performing more conditioning or warm up exercises at the beginning of the workout session. If the contextual user data is indicating that the user just completed a 5 K run prior to the workout session, the workout recommendation would lean toward performing fewer conditioning or warm up exercises at the beginning of the workout session. In another example, if the contextual user data is sleep quality data obtained from a web API of a wearable sleep tracking device and it is indicating that the user experienced a poor night of sleep the day before, the workout recommendation for the next day would lean towards performing a reduced volume of exercise movements to reduce risk of injury. FIG. 23 shows an example graphical representation illustrating a user interface 2300 for displaying adaptive training changes. The gamification engine 212 detects that the user has completed a 20 minute run prior to the workout from a fitness tracker device worn by the user. The gamification engine 212 modifies the workout session by removing warmup routines.

In some implementations, the gamification engine 212 receives contextual user data, such as nutritional data of the user from an API of a third-party diet-tracking and calorie counting application. The gamification engine 212 processes the data using a neural network to recommend food and supplement based recommendations. For example, if the nutritional data is indicative of the user not consuming enough protein to hit a target fitness goal, the recommendation would be a protein drink supplement or a post workout meal to purchase.

In some implementations, the gamification engine 212 tracks the user workout sessions on a day-to-day basis using the neural network for generating recommendations, such as next set of workout routines, heavier or lighter weights, purchase of additional exercise equipment, etc. In a first example, the gamification engine 212 receives the exercise performance statistics and metrics from the performance tracker 314, detects that a performance of a particular exercise movement is indicative of overtraining, and generates a recommendation to reduce a number of sets and/or repetitions of that particular exercise movement in the next workout session. In a second example, the gamification engine 212 recommends a mobility optimization workout as a next workout for hamstrings and/or quadriceps based on a determination that a heavy volume of barbell squat exercises were recently performed by the user. In a third example, the gamification engine 212 recommends a strength optimization workout as a next workout for antagonist muscles and/or other muscles that were undertrained in a recent workout session. In a fourth example, the gamification engine 212 recommends a conditioning optimization workout as a next workout to improve heart health, to rid the body of lactic acid, and to increase flexibility in between predominantly strength training workout sessions. In a fifth example, the gamification engine 212 recommends a purchase of additional exercise equipment (e.g., heavier weights) based on the exercise performance statistics and metric indicative of the user plateauing in strength training.

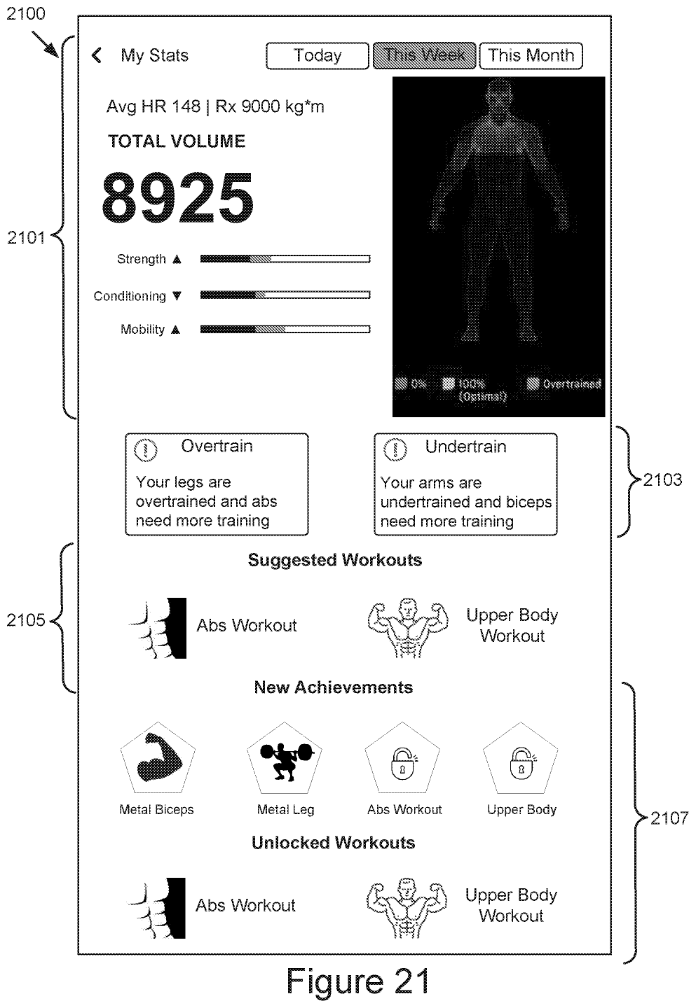

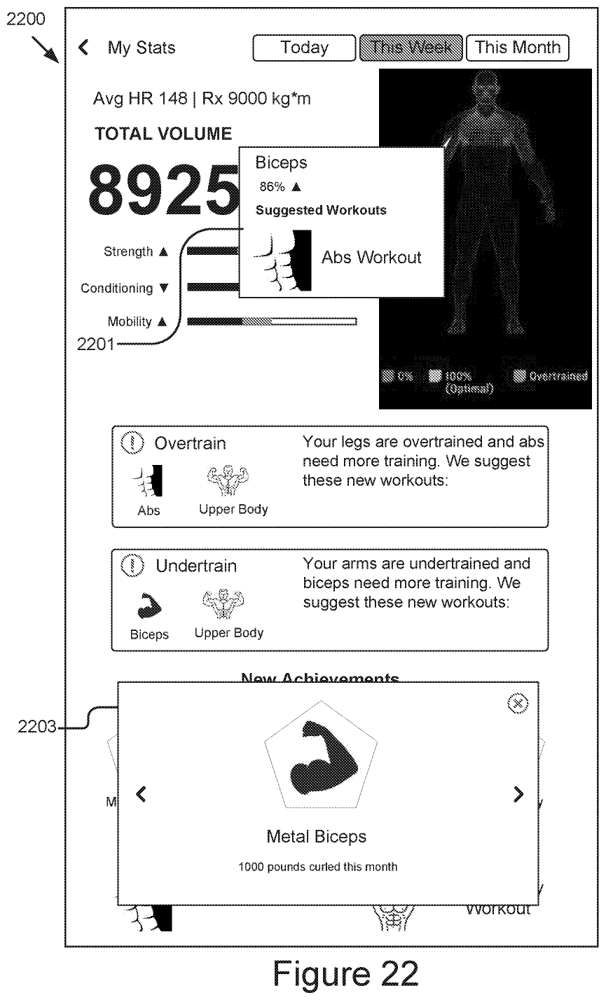

FIG. 21 shows another example graphical representation illustrating a user interface 2100 for displaying statistics relating to the user completion of an exercise workout session. The user interface 2100 depicts a display of statistics on the interactive screen of the interactive personal training device 108. The statistics is displayed in several portions. A first portion 2101 describes information about total volume, average heart rate, a progress bar for a mix of fitness goals, such as strength, conditioning, and mobility, and a representation of a virtual self of the user depicting a heat map for muscle groups that were overtrained (e.g., shown in red), undertrained (e.g., shown in blue), and/or optimally trained (e.g., shown in cyan). The progress bar is customizable to show progress over a period of time (e.g., a week, a month, a day, etc.). A second portion 2103 includes a textual notification of muscle groups that were overtrained and undertrained with useful information for user's consideration. A third portion 2105 includes a recommendation for next set of workouts based on the determination of overtrained and/or undertrained muscle groups in the concluded workout session. A fourth portion 2107 includes a set of achievements and workouts that were unlocked by the user based on their performance. FIG. 22 shows another example graphical representation illustrating a user interface 2100 for displaying statistics relating to the user completion of an exercise workout session. The user interface 2200 is depicting an alternative implementation of display of statistics in FIG. 21. For example, the user interface 2200 depicts a pop up notification 2201 with a suggested workout session in response to the user selecting a particular muscle group (undertrained or overtrained) on a representation of the virtual self of the user. The user interface 2200 also depicts a notification 2203 indicating that the user has earned an achievement badge for arm curling 1000 pounds in the past month.

The program enhancement engine 214 may include software and/or logic to provide functionality for enhancing one or more workout programs created by third-party content providers (e.g., via third-party servers 140) or users, such as personal trainers, coaches, pro-athletes, celebrities, boutique gyms, big box gyms, franchise health and fitness clubs, digital fitness content companies, etc. The program enhancement engine 214 provides access to the third-party content providers or users to create a set of exercise movements or workouts that may be enhanced using the feedback engine 208. For example, the feedback engine 208 analyzes the exercise movement in the created workout to enable a detection of repetition counting and the display of feedback in association with the exercise movement when it is performed by a user subscriber on the interactive personal training device 108. The program enhancement engine 214 receives a stream of sensor data including a video of a user (e.g., personal trainer) performing one or more repetitions of an exercise movement in the new workout program. The enhancement engine 214 analyzes the stream of sensor data including the video using the pose estimator 302 to estimate pose data relating to performing the exercise movement. The enhancement engine 214 instructs the user interface engine 216 to generate a user interface to present a dialogue box and receive from the user an input (e.g., ground truth) indicating the position, the angle, and the relative distance between the detected keypoints in a segment of the video containing a repetition of the exercise movement (e.g., barbell squat, shoulder press, etc.) from start to end. For example, the user uploads a video of the user performing a combination of a front squat movement and a standing overhead press movement. The user specifies the timestamps in the video segment that contain this new combination of exercise movement and sets conditions or acceptable thresholds for completing a repetition including angles and distance between keypoints, speed of movement, and range of movement. The enhancement engine 214 creates and trains a machine learning model for classifying the exercise movement using the user input as initial weights of the machine learning model and the video of the user performing the repetitions of the exercise movement. The enhancement engine 214 then applies this machine learning model on a plurality of videos of other users performing repetitions of this exercise movement from the new workout program. The enhancement engine 214 determines a performance of the machine learning model to classify the exercise movement in the plurality of videos. This performance data and associated manual labelling of incorrect classification is used to retrain the machine learning model to maximize the classification of the exercise movement and to provide feedback including repetition counting to user subscribers training with the new workout program.

The user interface engine 216 may include software and/or logic for providing user interfaces to a user. In some embodiments, the user interface engine 216 receives instructions from the components 202, 204, 206, 208, 210, 212, and 214, generates a user interface according to the instructions, and transmits the user interface for display on the interactive personal training device 108. In some implementations, the user interface engine 216 sends graphical user interface data to an application in the device 108 via the communication unit 241 causing the application to display the data as a graphical user interface.

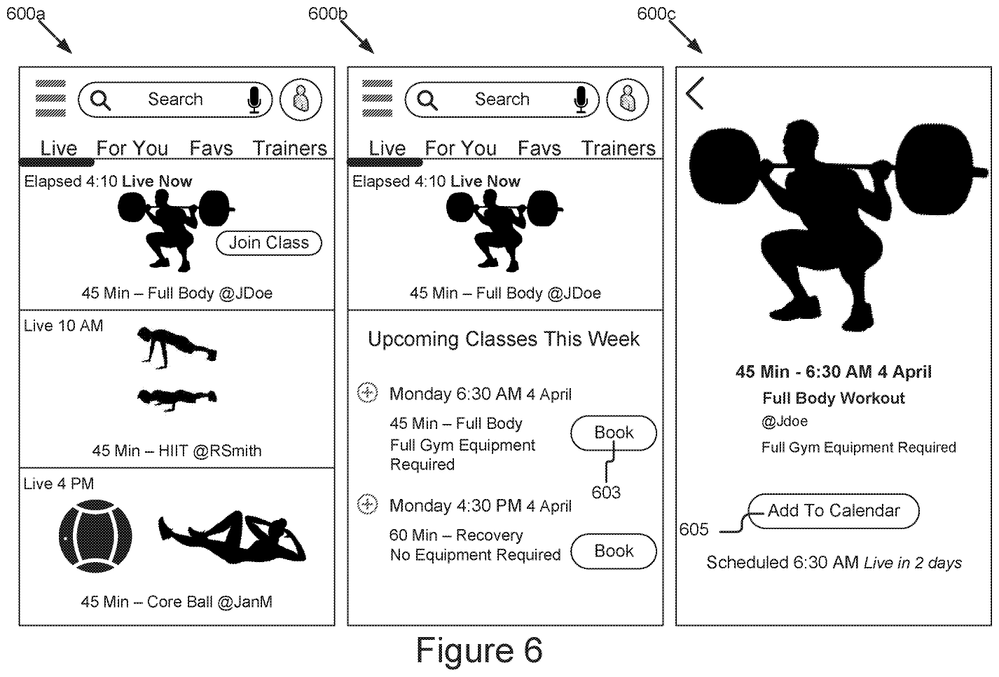

FIG. 6 shows example graphical representations illustrating user interfaces 600a-600c for adding a class to a user's calendar on the interactive personal training device 108. The user interface 600a depicts an interactive screen on the interactive personal training device 108 showing a list of live classes available for user selection. The user interface 600b depicts the interactive screen on the interactive personal training device 108 shown in response to the user selecting to view more information about the first listed live class. The user interface 600b shows information about upcoming classes for the selection and the user may click the button “Book” 603. The user interface 600c shows the class that has been booked for the user and the user may click the button “Add To Calendar” 605 to the add the class to his or her calendar.

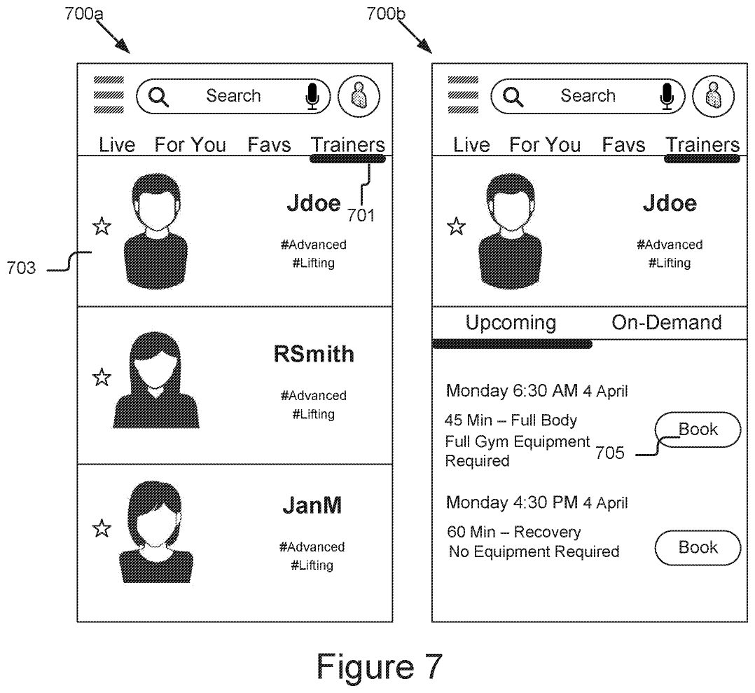

FIG. 7 shows example graphical representations illustrating user interfaces 700a-700b for booking a personal trainer on the interactive personal training device 108. The user interface 700a depicts an interactive screen on the interactive personal training device 108 showing a list of available personal trainers under the “Trainers” tab 701. The user interface 700b depicts the interactive screen on the interactive personal training device 108 that is shown in response to the user selecting to view more information about trainer JDoe 703. The user interface 700b shows the upcoming available personal training slots with the trainer and the user may click the button “Book” 705 to book a session with the personal trainer.

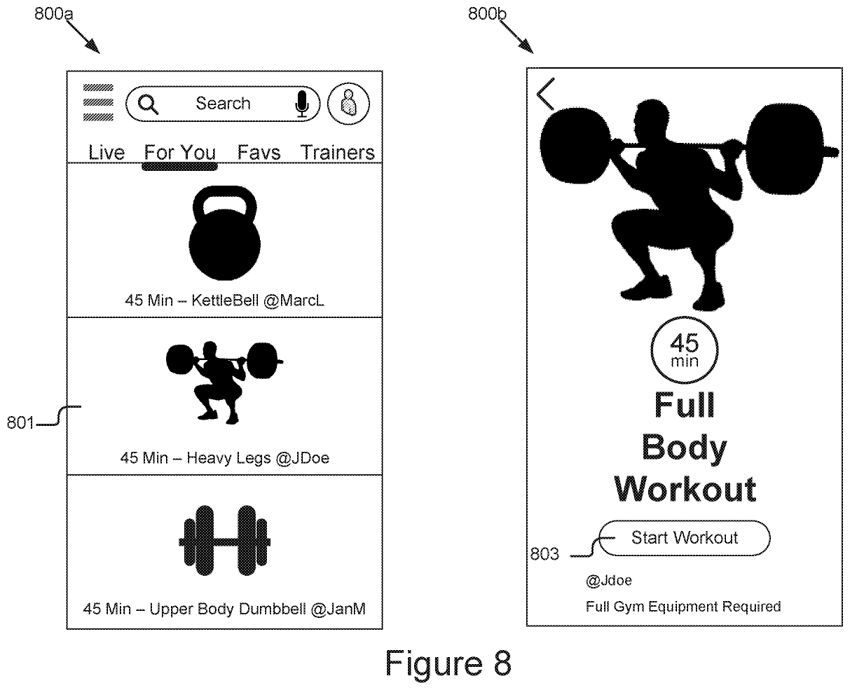

FIG. 8 shows example graphical representations illustrating user interfaces 800a-800b for starting a workout session on the interactive personal training device 108. The user interface 800a depicts an interactive screen on the interactive personal training device 108 showing a list of on-demand workout sessions available for user selection. The user interface 800b depicts the interactive screen on the interactive personal training device 108 that is shown in response to the user selecting a workout session 801. The user may click the button “Start Workout” 803 to begin the workout session.

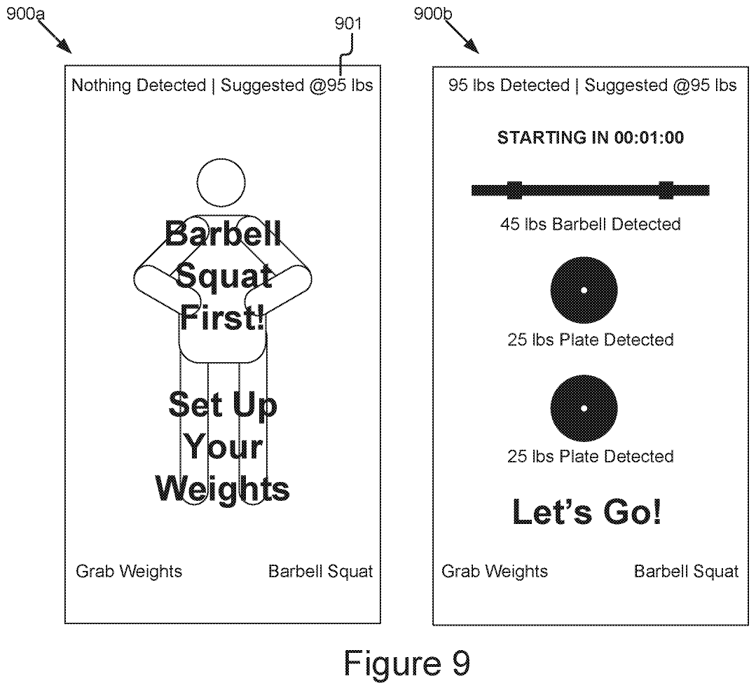

FIG. 9 shows example graphical representations illustrating user interfaces 900a-900b for guiding a user through a workout on the interactive personal training device 108. The user interface 900a depicts an interactive screen on the interactive personal training device 108 informing the user of an exercise movement barbell squat to perform and suggesting a weight 901 of 95 pounds for the exercise movement. As the user grabs the 45 pound barbell and two 25 pound plates, the user interface 900b depicts the interactive screen on the interactive personal training device 108 showing the detected weight equipment for the barbell squat. In some implementations, the equipment may be automatically detected on the interactive screen when the IMU sensor on the weight equipment communicates with the interactive personal training device 108 that the weight equipment has been picked up by the user.

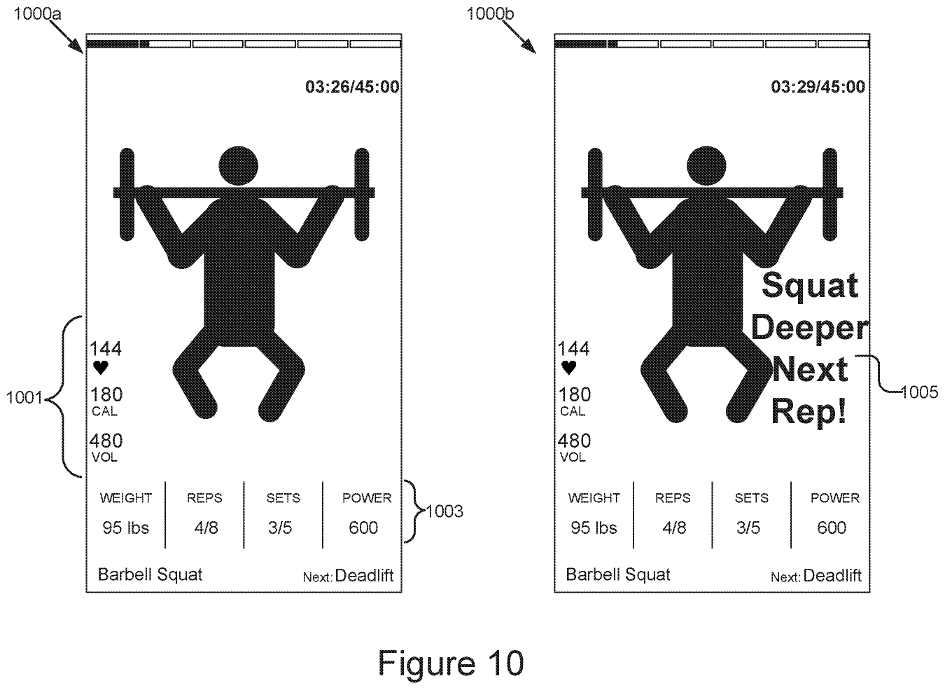

FIG. 10 shows example graphical representations illustrating user interfaces 1000a-1000b for displaying real time feedback on the interactive personal training device 108. The user interface 1000a depicts an interactive screen on the interactive personal training device 108 displaying real time feedback 1001 and 1003. The feedback 1001 includes a heart rate in beats per minute, calories burned, and the weight volume. The feedback 1003 includes the weight being moved in the exercise movement, the active count of number of repetitions completed, the active count of number of sets completed, and the power generated by the exercise movement. The user interface 1000b depicts the interactive screen on the interactive personal training device 108 displaying a recommendation 1005 for the user. The recommendation 1005 instructing the user to squat deeper for performing the squat exercise movement in the next repetition of the squat exercise movement.

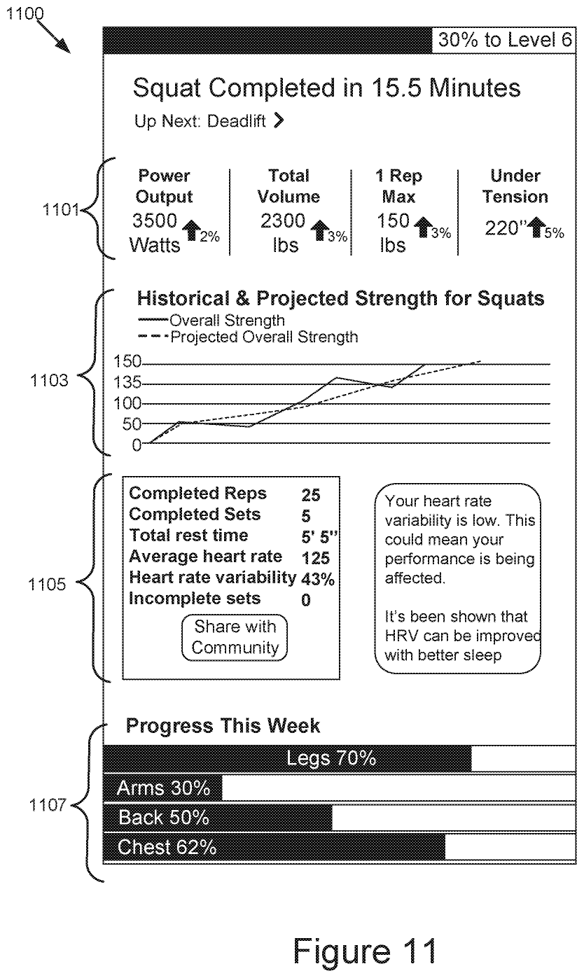

FIG. 11 shows an example graphical representation illustrating a user interface 1100 for displaying statistics relating to the user performance of an exercise movement upon completion. The user interface 1100 depicts a display of statistics on the interactive screen of the interactive personal training device 108. The statistics is displayed in several portions. A first portion 1101 describes information about power output, total volume, one-repetition maximum (1 Rep max), and time under tension for the exercise movement. A second portion 1103 includes a graph for plotting historical and projected strength gains for the exercise movement. A third portion 1105 includes a report on a completed set of exercise movement. The report includes a number of sets completed, a number of repetitions completed, total rest time, average heart rate, heart rate variability, etc. A fourth portion 1107 includes a progress bar showing a progress percentage for each muscle group.

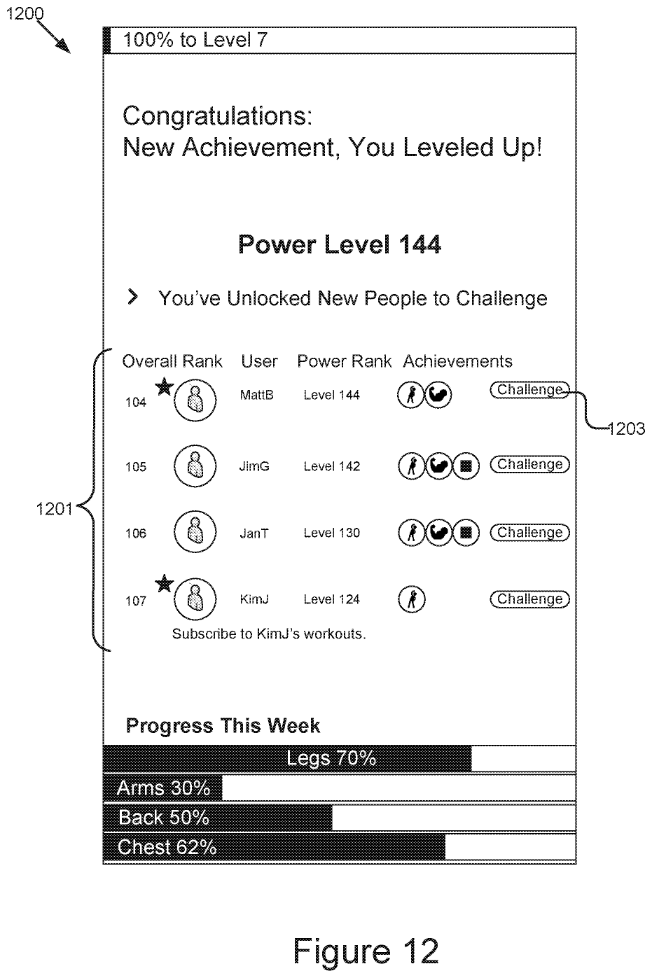

FIG. 12 shows an example graphical representation illustrating a user interface 1200 for displaying user achievements upon completion of a workout session. The user interface 1200 shows an achievement page for the user when the user has went up a power level upon completing exercise or workouts in the previous level. The user interface 1200 includes a list 1201 of peers in similar performance level and competence level as the user. The list 1201 includes an overall rank, name, power rank, and achievements of the peers. The user may choose to challenge a peer to compete with by selecting the button “Challenge” 1203. For example, the challenge may be on a select fitness category, such as a time-based fitness challenge, a strength challenge, a volume challenge, and an endurance challenge.

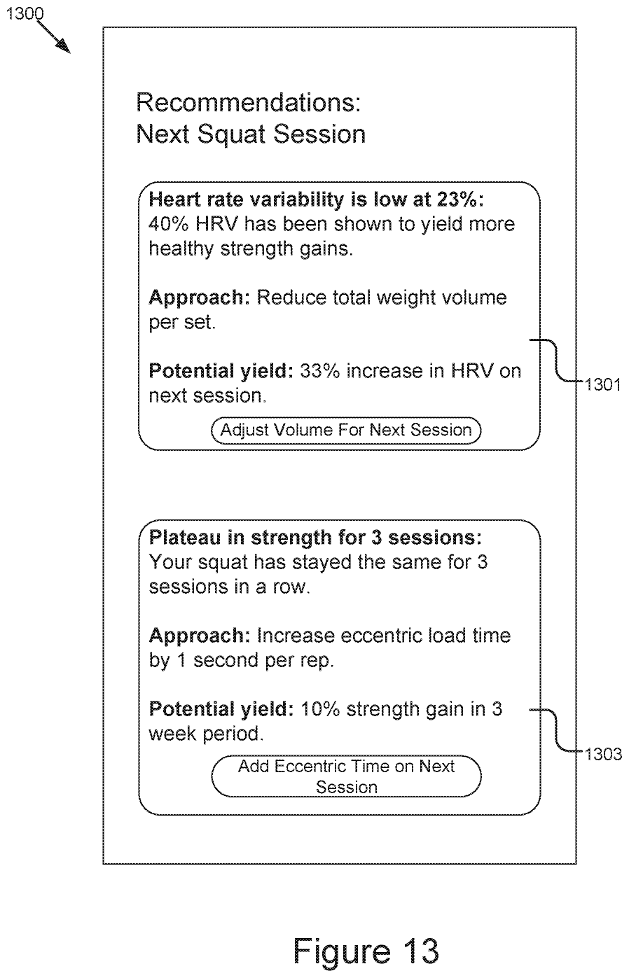

FIG. 13 shows an example graphical representation illustrating a user interface 1300 for displaying a recommendation to a user on the interactive personal training device 108. The user interface 1300 shows a first recommendation tile 1301 indicating an issue of low heart rate variability (HRV) in the user's performance, a recommendation to reduce total weight volume per set, and a potential yield indicating that this recommendation, if followed, will yield a 33% increase in HRV on the next session. The user interface 1300 shows a second recommendation tile 1303 indicating an issue of strength plateau for three workout sessions for the user, a recommendation to increase eccentric load time by one second per repetition, and a potential yield indicating that this recommendation, if followed, will yield a 10% strength gain in three week period.

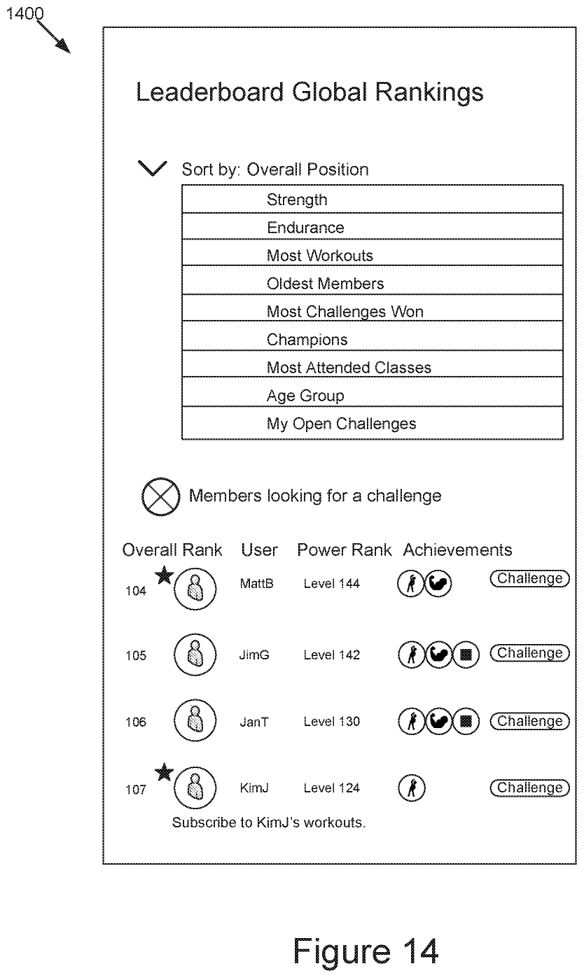

FIG. 14 shows an example graphical representation illustrating a user interface 1400 for displaying a leaderboard and user rankings on the interactive personal training device 108. The user interface 1400 shows a leaderboard and a user is able to select their preferred ranking category. The leaderboard may include a plurality of metrics, such as overall fitness, overall strength, overall endurance, most workouts, oldest members, most challenges won, similar performance (to the user), looking for a challenge, champions, most classes, age groups, sex, public challenges, my challenges, etc.

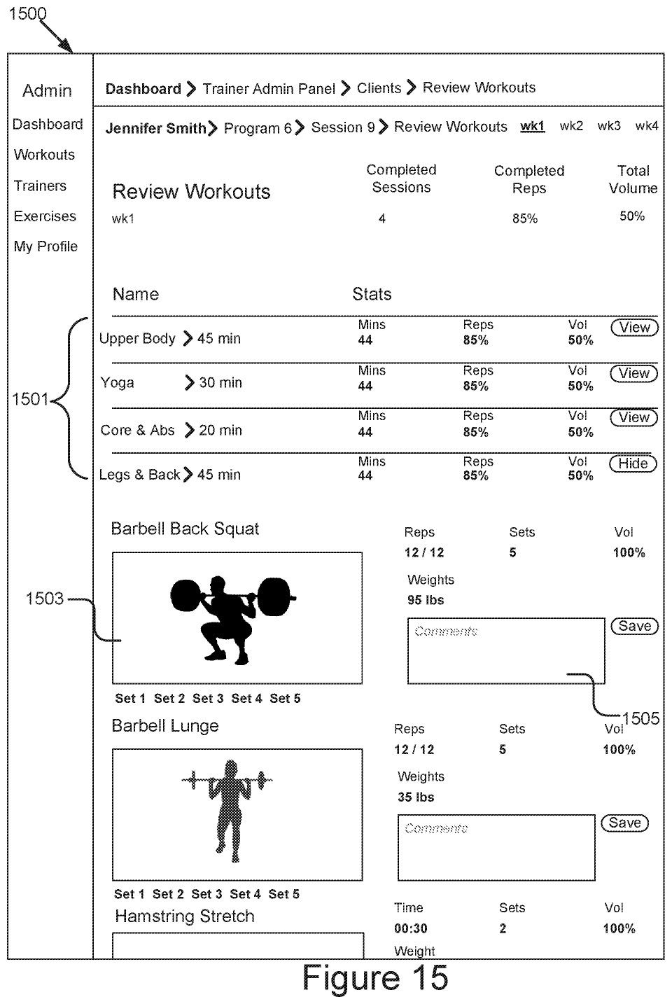

FIG. 15 shows an example graphical representation illustrating a user interface 1500 for allowing users (e.g., trainers) to plan, add, and review exercise workouts. The user interface 1500 shows an admin panel page for a trainer to review workouts done by clients. The statistics portion 1501 allows the trainer to view individual performance statistics for each workout session. The trainer may view a video 1503 of a client performing an exercise movement and leave comments providing feedback on the exercise movement in the comment box 1505.

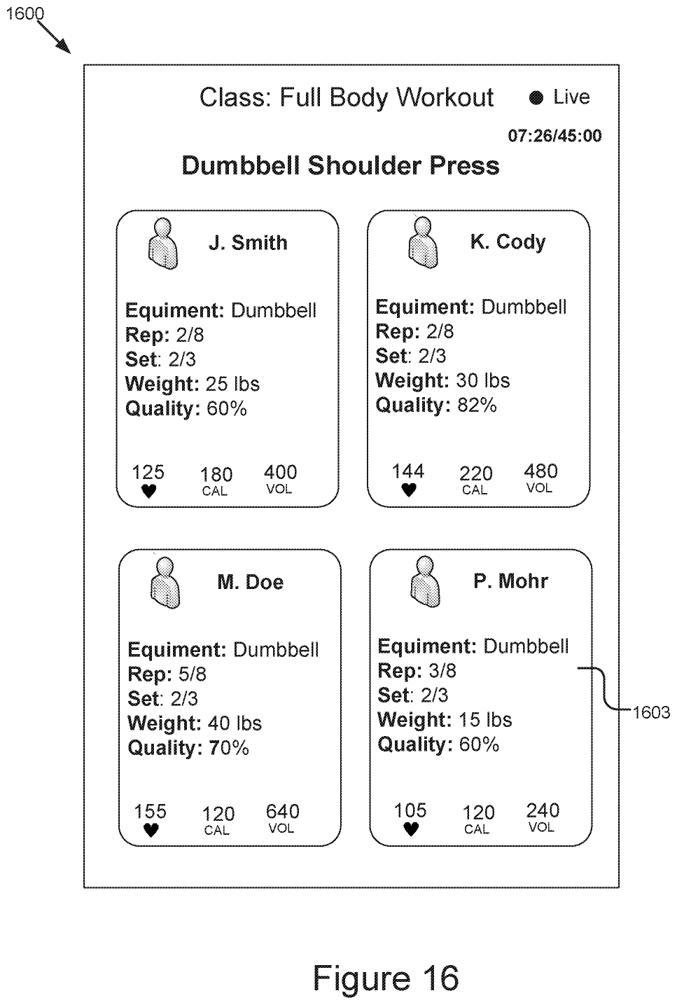

FIG. 16 shows an example graphical representation illustrating a user interface 1600 for a trainer to review an aggregate performance of a live class. The user interface 1600 shows collects each individual user performance for a number of users participating in a live class and provides a live view of the aggregate performance to a trainer situated remotely. The user interface 1600 includes a tile 1603 for each user indicating their use of a specific weight equipment, a weight in pounds of the specific weight equipment, a count of the number of repetitions done by the user, a count of the number of sets done by the user, a quality of the user's exercise movement repetition, heart rate, calories burned, weight volume, etc. The trainer gains the aggregate performance of the live class at a glance such that the trainer can better guide the class and praise or provide feedback to a particular user on their workout based on the data shown in their associated tile 1603.

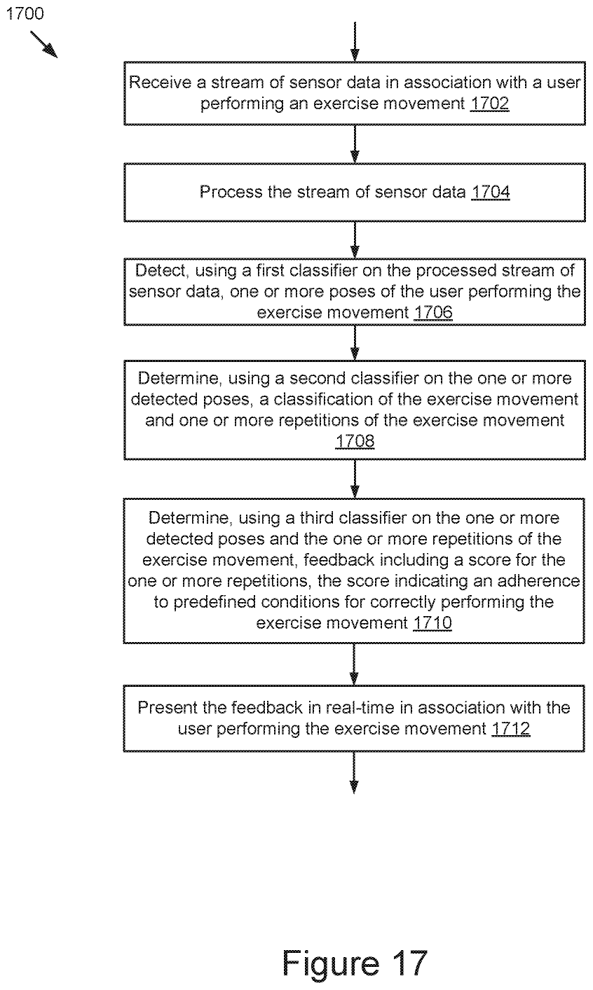

FIG. 17 is a flow diagram illustrating one embodiment of an example method 1700 for providing feedback in real-time in association with a user performing an exercise movement. At 1702, the data processing engine 204 receives a stream of sensor data in association with a user performing an exercise movement. For example, the stream of sensor data may be received over a period of time. At 1704, the data processing engine 204 processes the stream of sensor data. At 1706, the feedback engine 208 detects, using a first classifier on the processed stream of sensor data, one or more poses of the user performing the exercise movement. At 1708, the feedback engine 208 determines, using a second classifier on the one or more detected poses, a classification of the exercise movement and one or more repetitions of the exercise movement. At 1710, the feedback engine 208 determines, using a third classifier on the one or more detected poses and the one or more repetitions of the exercise movement, feedback including a score for the one or more repetitions, the score indicating an adherence to predefined conditions for correctly performing the exercise movement. At 1712, the feedback engine 208 presents the feedback in real-time in association with the user performing the exercise movement.

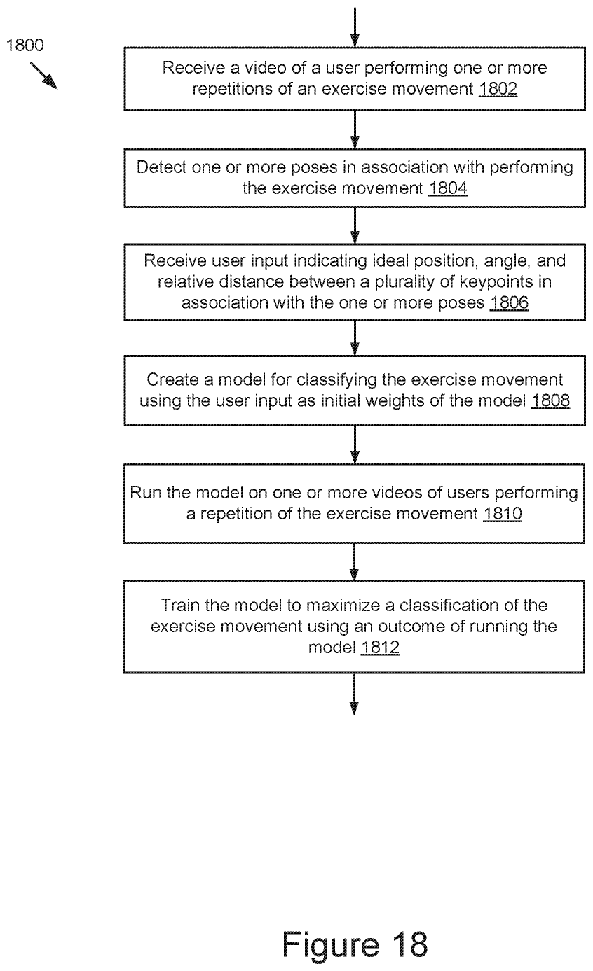

FIG. 18 is a flow diagram illustrating one embodiment of an example method 1800 for adding a new exercise movement for tracking and providing feedback. At 1802, the program enhancement engine 214 receives a video of a user performing one or more repetitions of an exercise movement. At 1804, the program enhancement engine 214 detects one or more poses in association with the user performing the exercise movement. At 1806, the program enhancement engine 214 receives user input indicating ideal position, angle, and relative distance between a plurality of keypoints in association with the one or more poses. At 1808, the program enhancement engine 214 creates a model for classifying the exercise movement using the user input as initial weights of the model. At 1810, the program enhancement engine 214 runs the model on one or more videos of users performing a repetition of the exercise movement. At 1812, the program enhancement engine 214 trains the model to maximize a classification of the exercise movement using an outcome of running the model.

Typically, existing connected fitness systems are vertically integrated. For example, the functionality offered by existing connected fitness systems are isolated from other platforms that provide fitness services (e.g., proprietary exercise equipment, independent exercise program content, unique user experience, etc.) to users. FIG. 1A implements an example cross-platform system for integrating with third-party content partners and service providers including one or more of fitness and sports brand companies, online fitness and nutrition companies, connected fitness systems, health tracking and wearable device companies, smart exercise equipment providers, and independent and/or company-based fitness content creators via the interactive personal training devices 108 and the personal training backend server 120 serving as a central hub. Each one of third-party partners (e.g., third-party servers 140) may have an API 136. The personal training application 110 implemented on one or more of the client device 130, the interactive personal training devices 108, and the personal training backend server 120 may communicate with the APIs of the third-party partners for connecting to their existing platforms and associated online services 111. This facilitates the personal training engine 202 of the personal training application 110 as described herein to request varied content including fitness programs from the third-party partners via the associated APIs and integrating the content into the user experience of the interactive personal training device 108 for the benefit of the user. Examples of third-party partners may include, but is not limited to, an independent Internet celebrity, trainer, or influencer with a count of followers, a pure play digital fitness content provider, and a fitness company (e.g., luxury gym, fitness franchise, health club, Yoga studio chain, big box gym, boutique gym, etc.). The third-party partners may sell their workout programs, fitness related media content, apparel, supplements, accessories, and other merchandize on a digital marketplace accessible to the users via one or more of the client device 130 and the interactive personal training device 108.

In some implementations, the personal training engine 202 may instantiate a channel for the platform provided by each one of the third-party partners. For example, users of the interactive personal training device 108 may be provided with an option to subscribe to a channel of a strength training digital content provider, a channel of a fitness franchise gym, a channel of an independent celebrity trainer, a channel of a Yoga studio chain, etc. In some implementations, when the user selects the channel of a particular third-party partner, the personal training engine 202 sends a request via the API and retrieves content, such as exercise workout programs from the associated platform of the third-party partner and presents them to the user. The personal training engine 202 in cooperation with the user interface engine 216 updates the user interface/user experience of the interactive personal training device 108 to match the user interface/user experience natively provided by the selected platform of the third-party partner. The personal training engine 202 may also facilitate a user to log into the selected platform on the interactive personal training device 108 using their login credentials associated with a membership account maintained with the third-party partner. Upon successful authentication, the personal training engine 202 directs the user to the home page of the selected platform on the interactive personal training device 108. In some implementations, the personal training engine 202 may categorize workout programs by trainers, workout type, fitness goals, etc. For example, each trainer may have a profile that a user may select to retrieve workout programs created by that trainer. In another example, a user may select a workout type including one or more of conditioning, strength, and mobility and retrieve suggested workout programs under a selected workout type. Partnership with third-party content and service providers allows the user to access a variety of new and interactive workout programs (e.g., enhanced by the program enhancement engine 214 as described herein) for free or on a fee-based subscription. The personal training engine 202 facilitates user purchase of workout programs, fitness related media content, apparel, supplements, accessories, and other merchandize made available by the third-party content and service providers using financial information, such as credit card information of the user stored in a profile 222 of the user.

As described herein, the personal training application 110 implemented on one or more of the client device 130, the interactive personal training devices 108, and the personal training backend server 120 uses sensors (e.g., IMU 132) embedded in the exercise equipment 134, the client device 130 (e.g., wearable activity trackers, smartwatches, smartphones, etc.), and machine learning-based three-dimensional image tracking and analysis to deliver exercise training programs from third-party partners to the users. The connected exercise equipment 134 and client devices 130 collect additional data and analytics for the personal training application 110 to perform its functionality as described herein. The third-party partners, such as a fitness franchise company may have numerous and experienced personal trainers. In some implementations, the personal training application 110 may virtually connect the personal trainers with the users of the interactive personal training device 108.

In some implementations, the data processing engine 204 of the personal training application 110 captures sensor data including a video of a user performing a workout in a session of a predetermined period of time. For example, the workout may be selected by the user based on a 45 minute High-Intensity Interval Training (HIIT) program provided by a third-party partner via the interactive personal training device 108. The feedback engine 208 analyzes the sensor data including a video of the user workout session and provides a feedback relating to one or more of exercise repetitions, weight equipment usage, adherence to proper form, user progress, and other statistics to the user. In some implementations, as part of the subscription to the third-party partner, the user may be entitled to have a video of them performing the exercise workout reviewed by a personal trainer for soliciting feedback. However, the personal trainer may be a trainer serving hundreds of clients. It may be impossible for the personal trainer to review the entire duration of the video and provide recommendations for each user. The data processing engine 204 processes the sensor data including the captured video of the workout session of a user using a machine learning algorithm or model as described herein. The data processing engine 204 creates a condensed or compressed video based on the data processing and analysis. In one example, the data processing engine 204 may compress the 45 minute recorded video of the workout session into a 4 minute compressed video. The data processing engine 204 may remove portions of the captured video that do not contain significant user activity, such as resting periods, picking up or putting down exercise equipment, interacting with the controls on the interactive personal training device 108, chatting, etc. and other data processing steps as described herein to generate the compressed video.

The data processing engine 204 may identify one or more segments in the compressed video that correspond to an exercise movement (e.g., barbell squat, dumbbell shoulder press, etc.) and attach metadata including repetition count, detected equipment weight, adherence score for proper form, and other statistics to the identified segments. For example, the data processing engine 204 identifies a 20 second segment of a user performing multiple repetitions of a dumbbell shoulder press, a 15 second segment of the user performing multiple repetitions of a bicep curl, etc. in the compressed video. Each one of the identified segments may be user selectable for review within the compressed video. The associated metadata for each one of the identified segment provides context including the number of sets completed, number of repetitions per set completed, weights used in an exercise routine, etc. The feedback engine 208 may generate performance statistics relating to the user in the captured video and attach them to the compressed video as metadata. For example, the feedback engine 208 determines average heart rate, calories burned, onset of fatigue, failure to complete a set of exercise movements in the workout, adherence score for the exercise movements, number of sets completed for each exercise movement, etc. as performance statistics and attaches them to the compressed video as metadata. The feedback engine 208 may flag a segment in the compressed video including an event of interest that could not be analyzed or classified by the machine learning model. The feedback engine 208 may deem the segment significant enough to require the personal trainer to review it. For example, the user may have performed a variation of an exercise movement that is not included in the prescribed workout program. The data processing engine 204 may send the compressed video to the third-party server 140 for a personal trainer to review. The personal trainer may review the compressed video and provide feedback to the user. The feedback may be in the form of a text message, a voice message, and/or video message. The feedback engine 208 may present the feedback to the user in association with the completed workout session on devices, such as the client device 130 and the interactive personal training device 108.

In some implementations, the program enhancement engine 214 of the personal training application 110 enables fitness content sourced from or uploaded by third-party partners to be enhanced for interactivity and made available to the users of the interactive personal training device 108 as described herein. For example, the enhancement may be in the form of enabling a detection of repetition counting and the display of feedback in association with a fitness program when it is performed by a user of the interactive personal training device 108. The program enhancement engine 214 receives a video of the training program created by the third-party partner and analyzes the video for enhancement. For example, the video may contain a personal trainer performing one or more repetitions of an exercise movement in the workout program. The feedback engine 208 estimates pose data including location of keypoints (e.g., knees, shoulder joints, elbows, etc.) in the analysis of the performance of the exercise movement in the video. The program enhancement engine 214 facilitates the personal trainer to set conditions or acceptable thresholds for successfully completing a repetition of an exercise movement with proper form or technique. For example, the acceptable thresholds defined by the personal trainer may include predefined angles and distance between the keypoints, speed of movement, range of movement, etc. The program enhancement engine 214 also facilitates the personal trainer to set up feedback to be relayed to the user if the thresholds or conditions are not appropriately met by the user during performance of the exercise movement. For example, the feedback may be displaying a graphical representation of a personal trainer ‘avatar’ correctly performing a squat exercise movement next to the 3D model of the user performing the same exercise movement for comparison. In another example, the feedback may be green tick mark displayed on the interactive screen for a perfect repetition of the exercise movement, a yellow tick mark displayed for an acceptable repetition of the exercise movement, and a red strike mark displayed for an incorrect form in the repetition of the exercise movement. In some implementations, the program enhancement engine 214 provides the third-party partner with a base acceptable thresholds prepopulated for a common set of exercise movements from exercise and fitness literature and associated feedback to be relayed to the user. The third-party partner has the ability to review and revise the base acceptable thresholds and associated feedback to match their training methodology or principles.

In some implementations, the personal training engine 202 enables third-party partners to monetize their fitness related content, such as training programs and merchandize. In one example, the program enhancement engine 214 allows a celebrity personal trainer to augment their fitness program offerings for repetition counting and displaying feedback as described herein. The fitness program may be a 16 week program made available to the users for a subscription fee (e.g., yearly subscription, monthly subscription, etc.). In another example, the personal training engine 202 facilitates a user of the interactive personal training device 108 to ‘Get a Look’ of the personal trainer in the workout program. The look of the personal trainer may be sponsored by a fitness apparel company or a supplement manufacturer. When the user selects to purchase, for example, the apparel worn by the trainer via the interactive personal training device 108, the personal training engine 202 places a purchase order with a website of the apparel company using credit card information of the user stored in the profile 222. A percentage cut of the transaction is awarded to the personal trainer. In yet another example, the data processing engine 204 may allow the personal trainer to review condensed video of their clients following the workout program for a fee (e.g., $15) at regular intervals. In another example, the gamification engine 212 facilitates a third-party partner, such as a luxury gym brand to upsell to the user a one-on-one in-person session with the personal trainer at a physical location.

In some implementations, the personal training engine 202 maintains a global user profile 222 of the user associated with the interactive personal training device 108. For example, the personal training engine 202 continually updates the global user profile 222 based on the actions performed by the user across the cross-platform and connected digital fitness system. For example, the user may select a channel of a strength training digital content provider and perform strength training workouts on the interactive personal training device 108 on three weekdays and select a channel of a Yoga studio chain and practice Yoga on the interactive personal training device 108 on the other weekdays. The personal training engine 202 tracks the user behavior including exercise workout routines of the user in the global user profile 222. The global user profile 222 provides a complete picture of the fitness regimen and practices of the user than the individual user profiles maintained by the different platform providers accessed via the interactive personal training device 108. The gamification engine 212 uses the global user profile 222 to maximize user engagement with the interactive personal training device 108 by recommending a next action that the user may want to perform. For example, the next action may be one or more of a trying a workout program, a third-party partner (e.g., aerobics) to sign on with, a product (e.g., supplements, apparel, etc.) to purchase, etc. Additionally, the global user profile 222 reduces the friction associated with opening a membership account with different third-party partner platforms on the interactive personal training device 108.

A system and method for tracking physical activity of a user performing exercise movements and providing feedback and recommendations relating to performing the exercise movements has been described. In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one embodiment above primarily with reference to software and particular hardware. However, the present invention applies to any type of computing system that can receive data and commands, and present information as part of any peripheral devices providing services.

Reference in the specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

Some portions of the detailed descriptions described above are presented in terms of algorithms and symbolic representations of operations on data bits within a computer memory. These algorithmic descriptions and representations are, in some circumstances, used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing”, “computing”, “calculating”, “determining”, “displaying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

Some embodiments can take the form of an entirely hardware embodiment, an entirely software embodiment or an embodiment containing both hardware and software elements. One embodiment is implemented in software, which includes but is not limited to firmware, resident software, microcode, etc.

Furthermore, some embodiments can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A data processing system suitable for storing and/or executing program code can include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description above. In addition, the techniques are not described with reference to any particular programming language. It will be appreciated that a variety of programming languages may be used to implement the teachings of the various embodiments as described herein.

The foregoing description of the embodiments has been presented for the purposes of illustration and description. It is not intended to be exhaustive or to limit the specification to the precise form disclosed. Many modifications and variations are possible in light of the above teaching. It is intended that the scope of the embodiments be limited not by this detailed description, but rather by the claims of this application. As will be understood by those familiar with the art, the examples may be embodied in other specific forms without departing from the spirit or essential characteristics thereof. Likewise, the particular naming and division of the modules, routines, features, attributes, methodologies and other aspects are not mandatory or significant, and the mechanisms that implement the description or its features may have different names, divisions and/or formats. Furthermore, as will be apparent to one of ordinary skill in the relevant art, the modules, routines, features, attributes, methodologies and other aspects of the specification can be implemented as software, hardware, firmware or any combination of the three. Also, wherever a component, an example of which is a module, of the specification is implemented as software, the component can be implemented as a standalone program, as part of a larger program, as a plurality of separate programs, as a statically or dynamically linked library, and/or in every and any other way known now or in the future to those of ordinary skill in the art of computer programming. Additionally, the specification is in no way limited to embodiment in any specific programming language, or for any specific operating system or environment. Accordingly, the disclosure is intended to be illustrative, but not limiting, of the scope of the specification, which is set forth in the following claims.

What is claimed is:

1. A computer-implemented method comprising:
receiving a selection of a fitness content provider from a user;
capturing sensor data including a video in association with the user performing a workout routine based on content from the fitness content provider;
analyzing, using a machine learning model, the captured sensor data including the video in association with the user performing the workout routine;
presenting a feedback to the user in association with the workout routine; and
generating a recommendation of a next action for the user.

2. The computer-implemented method of claim 1, further comprising:
responsive to receiving the selection of the fitness content provider from the user, sending a request via an application programming interface (API) of the fitness content provider to retrieve content; and
presenting the retrieved content in a user interface that natively matches that of the fitness content provider, the fitness content provider being a third-party service provider.

3. The computer-implemented method of claim 1, wherein analyzing the captured sensor data including the video in association with the user performing the workout routine further comprises:
identifying one or more of a number of repetitions of an exercise movement, a detected weight of an exercise equipment used in the exercise movement, a score indicating adherence to proper form, and user performance statistics in association with the user performing the workout routine.

4. The computer-implemented method of claim 1, wherein presenting the feedback to the user in association with the workout routine further comprises:
generating a three dimensional representation of an avatar based on the user;
translating user performance of the workout routine to a view of a heat map highlighting a part of a body on the avatar that was trained; and
presenting the three dimensional representation of the avatar including the view of the heat map.

5. The computer-implemented method of claim 4, wherein the view of the heat map highlighting the part of the body on the avatar indicates whether the part of the body was undertrained, overtrained, or optimally trained.

6. The computer-implemented method of claim 1, further comprising:
processing the captured sensor data including the video in association with the user performing the workout routine;
creating a condensed video based on processing the captured sensor data including the video;
identifying a segment in the condensed video corresponding to an exercise movement;
determining metadata based on analyzing the captured sensor data including the video; and
attaching the metadata to identified segment in the condensed video.

7. The computer-implemented method of claim 6, wherein generating the recommendation of the next action for the user further comprises:
sending the condensed to a personal trainer for review; and
receiving the recommendation of the next action for the user from the personal trainer.

8. The computer-implemented method of claim 6, wherein the metadata includes one or more of repetition count, detected equipment weight, adherence score for proper form, and performance statistics.

9. The computer-implemented method of claim 1, wherein the fitness content provider is one from a group of an independent personal trainer, a pure play digital fitness content provider, and a fitness company.

10. The computer-implemented method of claim 1, wherein the recommendation of the next action for the user is an adaptive workout to balance development in one or more fitness areas.

11. A system comprising:
one or more processors; and
a memory, the memory storing instructions, which when executed cause the one or more processors to:
receive a selection of a fitness content provider from a user;
capture sensor data including a video in association with the user performing a workout routine based on content from the fitness content provider;
analyze, using a machine learning model, the captured sensor data including the video in association with the user performing the workout routine;
present a feedback to the user in association with the workout routine; and
generate a recommendation of a next action for the user.

12. The system of claim 11, wherein the instructions further cause the one or more processors to:
responsive to receiving the selection of the fitness content provider from the user, send a request via an application programming interface (API) of the fitness content provider to retrieve content; and
present the retrieved content in a user interface that natively matches that of the fitness content provider, the fitness content provider being a third-party service provider.

13. The system of claim 11, wherein to analyze the captured sensor data including the video in association with the user performing the workout routine, the instructions further cause the one or more processors to:
identify one or more of a number of repetitions of an exercise movement, a detected weight of an exercise equipment used in the exercise movement, a score indicating adherence to proper form, and user performance statistics in association with the user performing the workout routine.

14. The system of claim 11, wherein to present the feedback to the user in association with the workout routine, the instructions further cause the one or more processors to:
generate a three dimensional representation of an avatar based on the user;
translate user performance of the workout routine to a view of a heat map highlighting a part of a body on the avatar that was trained; and
present the three dimensional representation of the avatar including the view of the heat map.

15. The system of claim 14, wherein the view of the heat map highlighting the part of the body on the avatar indicates whether the part of the body was undertrained, overtrained, or optimally trained.

16. The system of claim 11, wherein the instructions further cause the one or more processors to:
process the captured sensor data including the video in association with the user performing the workout routine;
create a condensed video based on processing the captured sensor data including the video;
identify a segment in the condensed video corresponding to an exercise movement;
determine metadata based on analyzing the captured sensor data including the video; and
attach the metadata to identified segment in the condensed video.

17. The system of claim 16, wherein to generate the recommendation of the next action for the user, the instructions further cause the one or more processors to:
send the condensed to a personal trainer for review; and
receive the recommendation of the next action for the user from the personal trainer.

18. The system of claim 16, wherein the metadata includes one or more of repetition count, detected equipment weight, adherence score for proper form, and performance statistics.

19. The system of claim 11, wherein the fitness content provider is one from a group of an independent personal trainer, a pure play digital fitness content provider, and a fitness company.

20. The system of claim 11, wherein the recommendation of the next action for the user is an adaptive workout to balance development in one or more fitness areas.
